(19) United States
(12) Patent Application Publication
(10) Pub. No.: US 2024/0206785 A1
(43) Pub. Date: Jun. 27, 2024

(54) Lancet for Use with a Dermal Patch System

(71) Applicant: Satio, Inc., Boston, MA (US)

(72) Inventors: Namal NAWANA, Weston, MA (US); Ziad Tarilk AL-SHAMSIE, San Diego, CA (US); Mike MONIZ, Boston, MA (US)

(73) Assignee: Satio, Inc., Boston, MA (US)

(21) Appl. No.: 18/597,513

(22) Filed: Mar. 6, 2024

Related U.S. Application Data
(63) Continuation-in-part of application No. 18/090,063, filed on Dec. 28, 2022.
Application No. 18/090,063 is a continuation-in-part of application No. 17/994,454, filed on Nov. 28, 2022, now Pat. No. 11,877,848.
Application No. 17/994,454 is a continuation of application No. 17/521,466, filed on Nov. 8, 2021, now Pat. No. 11,510,602.
Application No. 18/090,063 is a continuation-in-part of application No. 17/991,284, filed on Nov. 21, 2022.
Application No. 17/991,284 is a continuation-in-part of application No. 17/971,142, filed on Oct. 21, 2022.
Application No. 17/971,142 is a continuation-in-part of application No. 17/521,466, filed on Nov. 8, 2021, now Pat. No. 11,510,602.
Application No. 18/090,063 is a continuation-in-part of application No. 17/903,802, filed on Sep. 6, 2022.
Application No. 17/903,802 is a continuation of application No. 17/412,213, filed on Aug. 25, 2021, now Pat. No. 11,452,474.
Application No. 18/090,063 is a continuation-in-part of application No. 17/500,873, filed on Oct. 13, 2021, now Pat. No. 11,964,121.
(63) Continuation-in-part of application No. 18/090,026, filed on Dec. 28, 2022.
Application No. 18/090,026 is a continuation-in-part of application No. 17/903,802, filed on Sep. 6, 2022.
Application No. 17/903,802 is a continuation-in-part of application No. 17/500,873, filed on Oct. 13, 2021, now Pat. No. 11,964,121.
Application No. 17/500,873 is a continuation-in-part of application No. 17/994,454, filed on Nov. 28, 2022, now Pat. No. 11,877,848.
Application No. 17/994,454 is a continuation-in-part of application No. 17/971,142, filed on Oct. 21, 2022.
Application No. 17/971,142 is a continuation-in-part of application No. 17/991,284, filed on Nov. 21, 2022.
(63) Continuation-in-part of application No. 17/747,544, filed on May 18, 2022.
(63) Continuation-in-part of application No. 17/719,881, filed on Apr. 13, 2022.
(63) Continuation-in-part of application No. 17/412,205, filed on Aug. 25, 2021.
(60) Provisional application No. 63/190,700, filed on May 19, 2021.
(60) Provisional application No. 63/174,956, filed on Apr. 14, 2021.
(60) Provisional application No. 63/190,700, filed on May 19, 2021.
(60) Provisional application No. 63/174,956, filed on Apr. 14, 2021.
(60) Provisional application No. 63/190,700, filed on May 19, 2021.
(60) Provisional application No. 63/174,956, filed on Apr. 14, 2021.

(51) Int. Cl. A61B 5/15; A61F 13/84

(52) U.S. Cl. CPC A61B 5/150099; A61B 5/150206; A61B 5/150366; A61B 5/150755; A61F 13/84; A61F 2013/8473

(57) ABSTRACT

A dermal patch system includes a lancet with three needles and a cartridge that is configured to attach to the skin of a subject. The lancet is configured automatically move the three needles from an undeployed position to a deployed position when the lancet engages with the cartridge. The three needles are configured to puncture the skin of the subject to draw a physiological sample when in the deployed position.

### RELATED APPLICATIONS

The present application claims priority to U.S. application Ser. No. 17/719,881 filed on Apr. 13, 2022, which claims priority to U.S. Provisional Patent Application No. 63/174,956 filed on Apr. 14, 2021, U.S. application Ser. No. 17/412,205 filed on Aug. 25, 2021, which claims priority to U.S. Provisional Patent Application No. 63/190,700 filed on May 19, 2021 and U.S. Provisional Patent Application No. 63/174,956 filed on Apr. 14, 2021, U.S. application Ser. No. 17/747,544 filed on May 18, 2022, which claims priority to U.S. Provisional Patent Application No. 63/190,700 filed on May 19, 2021, U.S. application Ser. No. 17/903,802 filed on Sep. 6, 2022, U.S. application Ser. No. 18/090,026 filed on Dec. 28, 2022, U.S. application Ser. No. 17/971,142 filed on Oct. 21, 2022, U.S. application Ser. No. 17/991,284 filed on Nov. 21, 2022 and U.S. application Ser. No. 18/090,063 filed on Dec. 28, 2022, which are hereby incorporated by reference herein in their entireties.

### TECHNICAL FIELD

The present teachings are generally directed to a lancet for use with a cartridge of a dermal patch system (also referred to as “a dermal patch” herein).

### BACKGROUND

Several diagnostic tests may be performed by analyzing a physiological sample from a subject. In order to obtain the physiological sample, a subject may have to travel to a diagnostic lab, where a medical professional obtains the sample with a standard syringe. This process may be time consuming and burdensome to the subject.

### SUMMARY

Aspects of the present disclosure address the above-referenced problems and/or others.

In one aspect, a dermal patch system includes a lancet including one, two or three or four, or more needles, and a cartridge configured to attach to the skin of a subject. The lancet is configured automatically move the needle(s) from an undeployed position to a deployed position when the lancet engages with the cartridge. The needle(s) are configured to puncture the skin of the subject to draw a physiological sample when in the deployed position. In some embodiments, the cartridge is configured to store the drawn physiological sample. In some embodiments, when three needles are used, the three needles can be equally spaced from one another. In these embodiments, tips of the three needles form an equilateral triangle. In some embodiments, the lancet includes a housing, and the needle(s) are disposed within the housing when in the undeployed position and extend beyond the housing when in the deployed position. In some embodiments, the needle(s) are configured to automatically retract into the housing after puncturing the skin. In some embodiments, the lancet further includes: an injection spring configured to move the needle(s) to the deployed position, and a retraction spring configured to retract the needle(s) into the housing. In some embodiments, the lancet includes a mechanism for maintaining the retraction spring is a compressed state when the needle(s) are in the undeployed position and is configured to expand to retract the needle(s). In some embodiments, the lancet further includes a plurality of deformable tabs configured to compress the retraction spring when the needle(s) are in the undeployed position. In some embodiments, the lancet further includes a needle platform that carries the needle(s) from the unemployed position to the deployed position. The needle platform is configured to deform the tabs and, when deformed, the tabs allow the retraction spring to expand. In some embodiments, the needle platform is configured to break the tabs.

In another aspect, a method for obtaining a physiological sample from a subject includes affixing a cartridge of a dermal patch system to the skin of a subject, and engaging a lancet with the cartridge, wherein the lancet includes needle(s). Engaging the lancet with the cartridge automatically moves the needle(s) from an undeployed position to a deployed position thereby drawing a physiological sample. In some embodiments, the method also includes storing physiological sample within the cartridge. In some embodiments, three needles are equally spaced from one another. In some embodiments, the tips of the three needles form an equilateral triangle. In some embodiments, the method also includes retracting the needle(s) into the lancet, and removing the cartridge from the skin of the subject. In some embodiments, the lancet automatically retracts the needle(s). In some embodiments, the lancet includes a first spring that moves the needle(s) to the deployed position and a second spring that retracts the needle(s). In some embodiments, the method also includes deforming a portion of the lancet whole moving needle(s) to the deployed position. In another embodiments, the method also includes deforming a portion of the lancet allows the second spring to retract the needle(s).

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIG. 1 depicts a dermal patch system in accordance with an exemplary embodiment of the present disclosure;

FIG. 2 depicts a lancet with three needles in accordance with an exemplary embodiment of the present disclosure;

FIG. 3 is a cross sectional view of the lancet, wherein the lancet is in an undeployed position in accordance with an exemplary embodiment of the present disclosure;

FIG. 4 is a cross sectional view of a housing of the lancet in accordance with an exemplary embodiment of the present disclosure;

FIG. 5 is a cross sectional view of a cap of the lancet in accordance with an exemplary embodiment of the present disclosure;

FIG. 6 depicts a side of an inner sleeve of the lancet in accordance with an exemplary embodiment of the present disclosure;

FIG. 7 depicts another side of the inner sleeve of the lancet in accordance with an exemplary embodiment of the present disclosure;

FIG. 8 is a cross sectional view of the inner sleeve of the lancet in accordance with an exemplary embodiment of the present disclosure;

FIG. 9 depicts a side of a needle platform of the lancet in accordance with an exemplary embodiment of the present disclosure;

FIG. 10 depicts another side of the needle platform in accordance with an exemplary embodiment of the present disclosure;

FIG. 11 is a cross sectional view of a spring sleeve of the lancet in accordance with an exemplary embodiment of the present disclosure;

FIG. 12 is a top view of a cover of a cartridge of the dermal patch system in accordance with an exemplary embodiment of the present disclosure;

FIG. 13 is a cross sectional view of the cover in accordance with an exemplary embodiment of the present disclosure;

FIG. 14 is a top view of a base of the cartridge of the dermal patch system in accordance with an exemplary embodiment of the present disclosure;

FIG. 15 is a cross sectional view of a lancet receiving element of the base in accordance with an exemplary embodiment of the present disclosure;

FIG. 16 is a cross sectional view of the base in accordance with an exemplary embodiment of the present disclosure;

FIG. 17 schematically depicts a physiological sample well, a physiological sample channel, and a sample collection reservoir of the base in accordance with an exemplary embodiment of the present disclosure;

FIG. 18 is a cross sectional view of the lancet engaged with the base, wherein the needle platform is in a released position in accordance with an exemplary embodiment of the present disclosure;

FIG. 19 is a cross sectional view of the lancet, wherein the needle platform is in a released position in accordance with an exemplary embodiment of the present disclosure;

FIG. 20 is a cross sectional view of the lancet transitioning to a deployed position in accordance with an exemplary embodiment of the present disclosure;

FIG. 21 is a cross sectional view of the lancet engaged with the base, wherein the lancet is in a deployed position in accordance with an exemplary embodiment of the present disclosure;

FIG. 22 is a cross sectional view of the lancet, wherein the lancet is in a deployed position in accordance with an exemplary embodiment of the present disclosure;

FIG. 23 is a cross sectional view of the lancet engaged with the base, wherein the lancet is in a retracted position in accordance with an exemplary embodiment of the present disclosure;

FIG. 24 is a cross sectional view of the lancet, wherein the lancet is in a retracted position in accordance with an exemplary embodiment of the present disclosure;

FIG. 25 depicts a dermal patch system in accordance with an exemplary embodiment of the present disclosure;

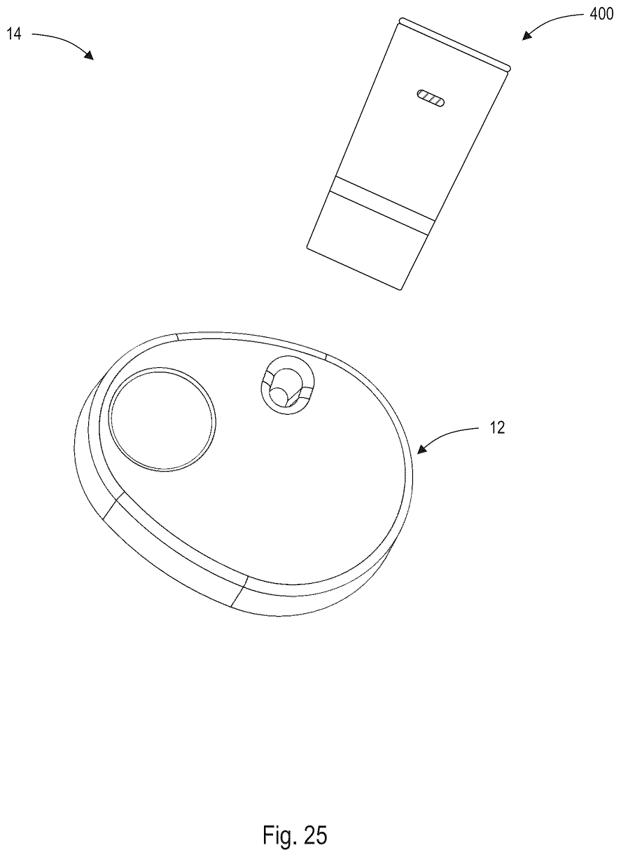

FIG. 26 shows the lancet with a sensor formed therein;

FIG. 27 shows a fingerprint database in accordance with an exemplary embodiment of the present disclosure;

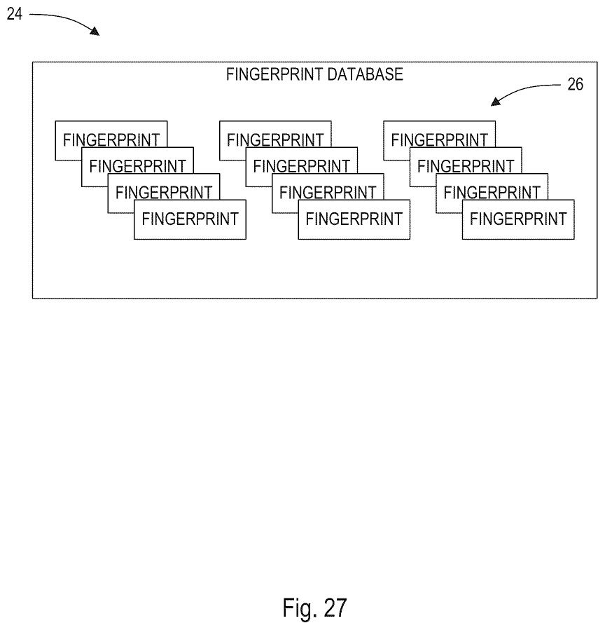

FIG. 28 is a cross sectional view of the lancet, wherein the lancet is in a fully retracted position in accordance with an exemplary embodiment of the present disclosure;

FIG. 29 is a cross sectional view of the lancet, wherein the lancet is in a fully retracted position in accordance with an exemplary embodiment of the present disclosure;

FIG. 30 shows a computer system connected to a fingerprint data base in accordance with an exemplary embodiment of the present disclosure;

FIG. 31 shows a computer system connected to a biometric data base in accordance with an exemplary embodiment of the present disclosure;

FIG. 32 shows a computer system connected to an EMR data base in accordance with an exemplary embodiment of the present disclosure;

FIG. 33 shows a method for obtaining a physiological sample from a subject in accordance with an exemplary embodiment of the present disclosure;

FIG. 34 is a flow chart showing a method for obtaining a physiological sample from a subject in accordance with an exemplary embodiment of the present disclosure;

FIG. 35 shows a computer system in accordance with an exemplary embodiment of the present disclosure;

FIG. 36 shows a cloud computing environment in accordance with an exemplary embodiment of the present disclosure.

### DETAILED DESCRIPTION

The present disclosure generally relates to a dermal patch system (which may also be referred to as a “dermal patch”) that may be utilized to collect and optionally store a physiological sample.

In various aspects, the present teachings address an unmet need for devices that allow efficient, easy and reliable extraction of biological samples from a subject.

In various embodiments, a dermal patch system is disclosed that allows collecting a physiological sample and storing the collected physiological sample, e.g., on a sample collection pad and/or a reservoir provided on the dermal patch system. Dermal patch systems disclosed herein may allow for the collection and analysis of a physiological sample in a variety of environments (e.g., at home, in the field, in a medical facility, etc.).

The term “about,” as used herein, denotes a deviation of at most 10% relative to a numerical value. For example, about 100 μm means in the range of 90 μm-110 μm.

The term “substantially,” as used herein, refers to a deviation, if any, of at most 10% from a complete state and/or condition.

The term “majority,” as used herein, refers to more than 50% of an object or substance.

The term “subject” as used herein refers to a human subject or an animal subject (i.e., chicken, pig, cattle, dog, cat, etc.).

The term “transparent,” as used herein, indicates that light can substantially pass through an object (e.g., a window) to allow visualization of a material disposed behind the object. For example, in some embodiments, a transparent object allows the passage of at least 70%, or at least 80%, or at least 90% of visible light therethrough.

The term “needle” as used herein, refers to a component with a pointed tip that is configured to pierce an outer surface of an element (e.g., skin of a subject) to provide a passageway through which a physiological fluid can be extracted. One, two, three, four, or more needles can be used.

The present disclosure generally relates to a device, which is herein also referred to as a dermal patch or a dermal patch system, for collecting a physiological sample (e.g., bodily fluids such as blood, interstitial fluids, etc.) from a subject. In some embodiments discussed below, such a dermal patch system can include a cartridge that can be affixed to a subject's skin (e.g., via an adhesive layer) and a separate lancet that can be engaged with the cartridge to puncture the skin, thereby providing a passageway for extracting the physiological sample. As discussed in more detail below, the lancet can include a housing in which at least one needle that is configured for puncturing the skin is disposed. The lancet can be transitioned between at least three states. In a first state (which may be referred to as an undeployed or a locked state) the lancet retains the needle(s) within the lancet housing when the lancet is not engaged with the cartridge. In a second state (which may be referred to as a deployed, an unlocked state, or a released state) the lancet allows the needle(s) to be deployed to allow the needle(s) to exit the housing, so as to be available for puncturing the skin in response to engagement of the lancet with the cartridge. In a third state (which may be referred to as a retracted state), subsequent to the deployment of the needle(s), the lancet automatically moves the needle(s) from the deployed position back into the lancet housing. In other words, the engagement of the lancet with the cartridge automatically transitions the needle(s) from the locked state to the unlocked state, and to the retracted state.

In some embodiments, as discussed in more detail below, the lancet includes an inner sleeve with a plurality of deformable tabs and a spring sleeve. In a locked state, the deformable tabs can apply a pressure to the spring sleeve to maintain the spring sleeve in a first position in which the spring sleeve compresses the lower spring into a compressed state and prevents the lower spring from expanding.

When the lancet is engaged with the cartridge, the inner sleeve moves from a first position to a second position. In the first position, the inner sleeve retains a needle platform that supports the needle(s) in an undeployed position. The lancet also includes an upper spring that is coupled to the needle platform. In the undeployed position, the needle platform compresses the upper spring and prevents the upper spring form expanding. In the second position, the inner sleeve moves vertically upward, which releases the needle platform thereby allowing the needle platform to move to a deployed position. When released, the upper spring is allowed to expand thereby applying a force to the needle platform, which causes the movement of the needle platform such that the needle(s) extend beyond the housing of the lancet and become available for puncturing the skin.

Furthermore, this force causes the needle(s) (which are coupled to the needle platform) to travel with sufficient velocity to puncture the skin of a subject. As the needle platform travels to the deployed position, the needle platform contacts the deformable tabs and causes the tabs to break or deflect. When broken (or deflected), the tabs no longer contact the spring sleeve, or apply a lower force onto the spring sleeve, thereby allowing the lower spring to expand and move the spring sleeve vertically upward from a first position to a second position, which in turn moves the needle spring vertically upward such that the needle(s) are retracted into the housing of the lancet.

In this manner, the lancet remains safe before it is engaged with the cartridge as the needle(s) in the lancet cannot be deployed when the lancet is not engaged with the cartridge. Furthermore, in this manner, the lancet remains safe after drawing a physiological sample as the needle(s) automatically retract back into the lancet's housing after being deployed.

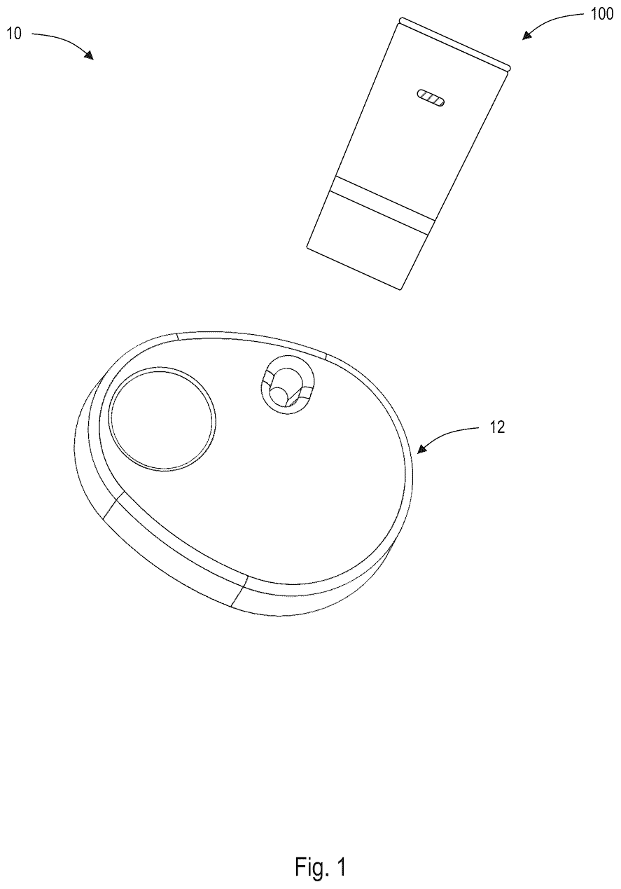

Referring now to FIG. 1, a dermal patch system 10 is shown in accordance with an exemplary embodiment. In this embodiment, the dermal patch system 10 includes a cartridge 12 that can be affixed to a subject's skin and a lancet 100 that can be engaged with the cartridge 12.

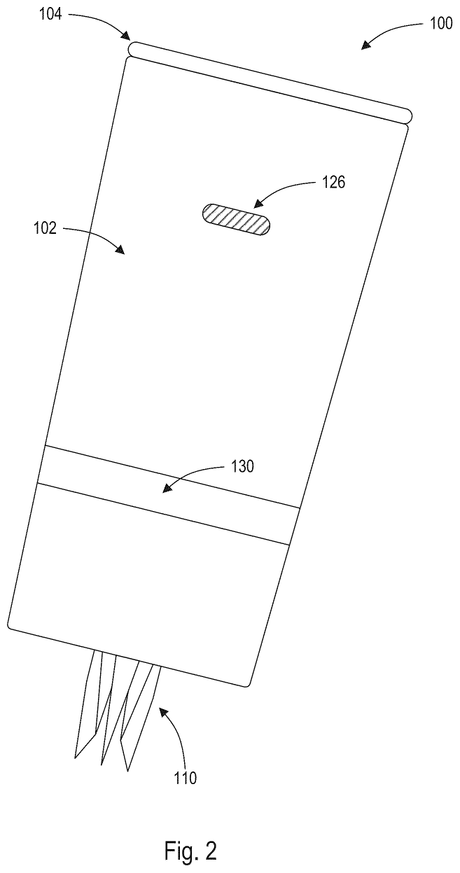

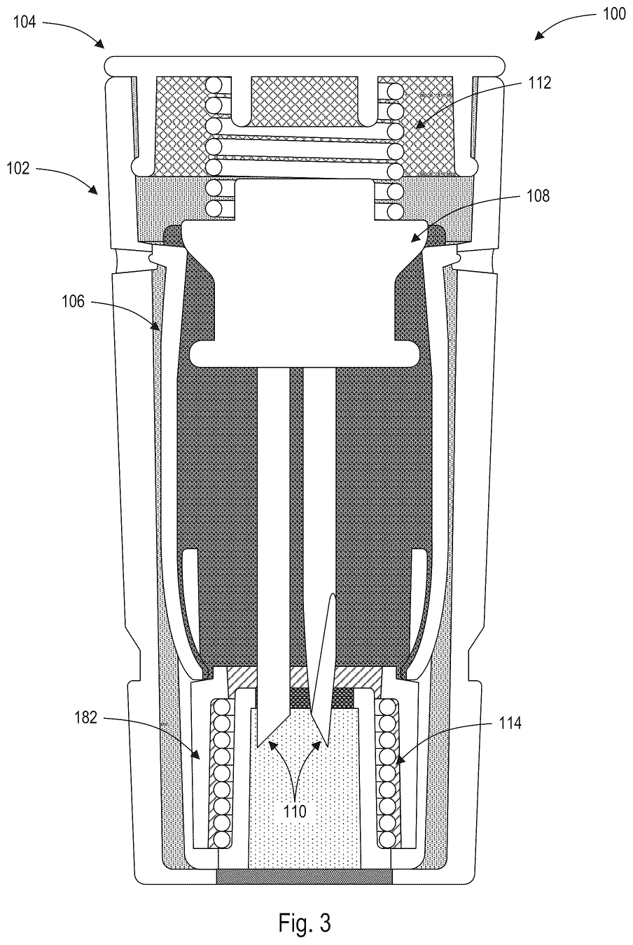

Referring to FIGS. 2 and 3, a cross sectional view of the lancet 100 is shown in accordance with an exemplary embodiment. The lancet 100 include a housing 102 and a cap 104 that is coupled to the housing 102. The lancet extends longitudinally along an X-axis (FIG. 3) between a proximal end defined by the cap 104 and a distal end defined by the housing 102.

The housing 102 and the cap 104 define an inner volume of the lancet 100 in which various components of the lancet 100 are disposed. The lancet 100 further includes an inner sleeve 106 and a needle platform 108 that are disposed within the inner volume of the lancet 100. The needle platform 108 supports a plurality of needles 110, specifically three needles 110 in this embodiment. Although three needles are shown in FIGS. 2 and 3, as previously mentioned, one, two, three or four, or more needles can be used.

While needle platform 108 is depicted as supporting three needles 110 (FIG. 2), in other embodiments, the needle platform 108 can support more or less needles 110 (e.g., 1, 2, 4, 5, needles etc.). However, it has been discovered that the use of three needles is particularly advantageous in drawing blood from a subject. Further, in this embodiment, the needles have slanted ends and the bottom ends of the needles facing away from one another.

The lancet 100 further includes an injection spring (also referred to as an upper spring) 112 and a retraction spring (also referred to as a lower spring) 114 that can move the needle platform 108.

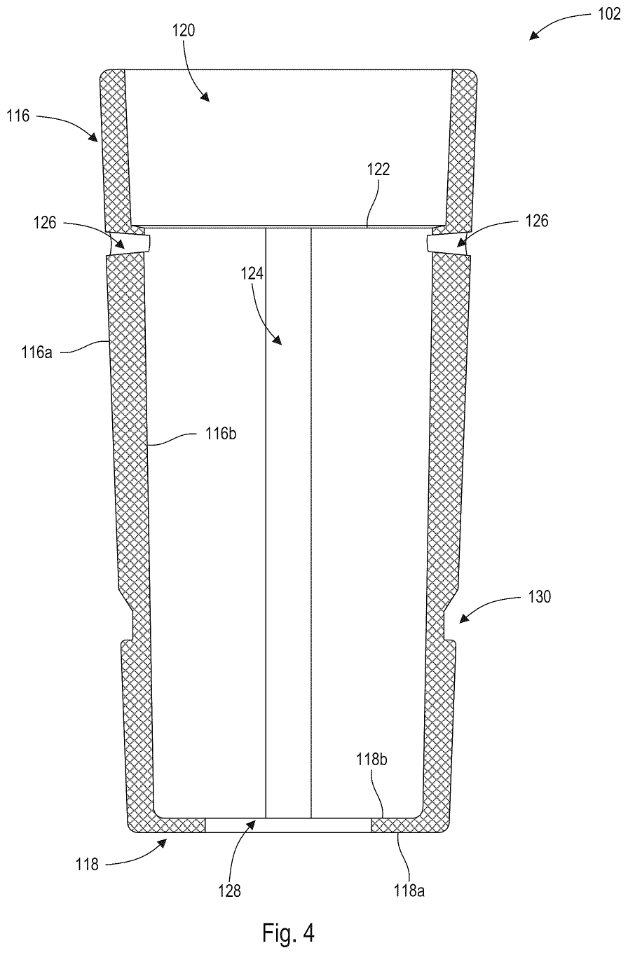

With particular reference to FIG. 4, the housing 102 includes a side wall 116 and a bottom wall 118. The side wall 116 includes an outer surface 116a and an opposed inner surface 116b. The bottom wall 118 includes an outer surface 118a and an opposed inner surface 118b. The side wall 116 extends vertically from the bottom wall 118. In this embodiment, the side wall 116 is substantially cylindrical in shape and the bottom wall 118 has a substantially circular cross-section. The side wall 116 and the bottom wall 118 are generally symmetric about a longitudinal axis (Y-axis) of the lancet 100.

The inner surface 116b of the side wall 116 and the inner surface 118b of the bottom wall 118 define an inner volume 120. The side wall 116 is tapered such that its inner diameter increases in a direction from its distal end to its proximal end such that the generally the lancet housing exhibits a wider upper portion relative to its lower portion. Further, the inner surface 118b of the upper portion defines a ledge 122.

The inner surface 116b also defines a first and second longitudinal groove 124 that are opposed from one another (only one of which is shown in the cross section in FIG. 4). The grooves 124 extend between the ledge 122 and the inner surface 118b of the bottom wall 118.

The side wall 116 defines openings 126 that extend through the side wall 116. That is, the openings 126 extend between the outer surface 116a and the inner surface 116b of the side wall 116. The bottom wall 118 defines an aperture 128 that extends through the bottom wall 118. That is, the aperture 128 extends between the outer surface 118a and the inner surface 118b of the bottom wall 118. As will be discussed in further detail herein, when the lancet 100 is activated via engagement with the cartridge 12, the needle(s) of the lancet 100 extend through the aperture 128 for puncturing a subject's skin to draw a physiological sample. The outer surface 116b of the side wall 116 further defines an outer groove 130. As will be discussed in further detail herein, a portion of the cartridge 12 engages with the outer groove 130 to couple the lancet 100 to the cartridge 12.

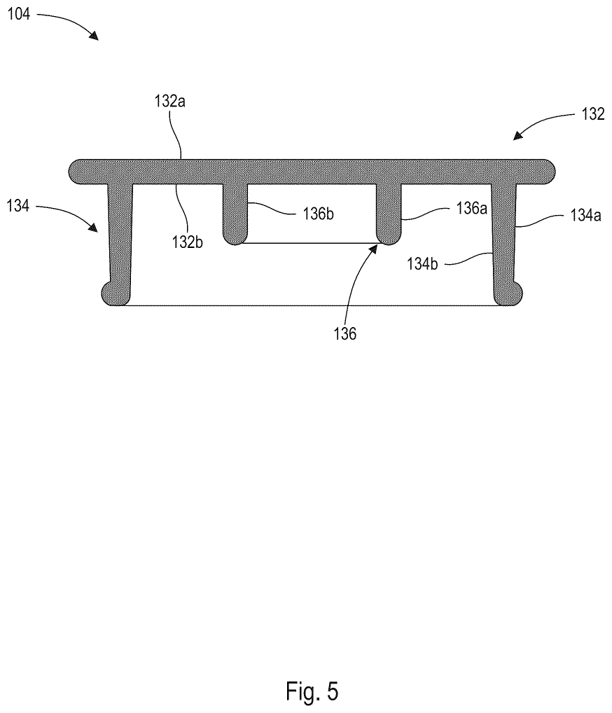

Referring now to FIG. 5, the inner cap 104 is shown in accordance with an exemplary embodiment. The cap 104 includes a top wall 132 with an outer surface 132a and an opposed inner surface 132b. The cap 104 also includes a side wall 134 with an outer surface 134a and an opposed inner surface 134b. The top wall 132 extends substantially horizontally from and perpendicular to the side wall 134. The side wall 134 extends substantially vertically from and perpendicular to the top wall 132. The top wall 132 and the side wall 134 have generally circular cross-sectional profiles and are concentric with one another. The cap 104 also includes an inner cylinder 136 with an outer surface 136a and an opposed inner surface 136b. The inner cylinder 136 extends vertically from and perpendicular to the top wall 132. The inner cylinder 136 is generally concentric with the top wall 132 and the side wall 134. The inner cylinder 136 extends substantially vertically from and perpendicular to the top wall 132.

When the cap 104 is coupled to the housing 102 the side wall 134 extends into the inner volume 120 of the housing 102 and at least a portion of the side wall 134 contacts the inner surface 116b of the side wall 116 such that the cap 104 couples to the housing 102 via an interference fit.

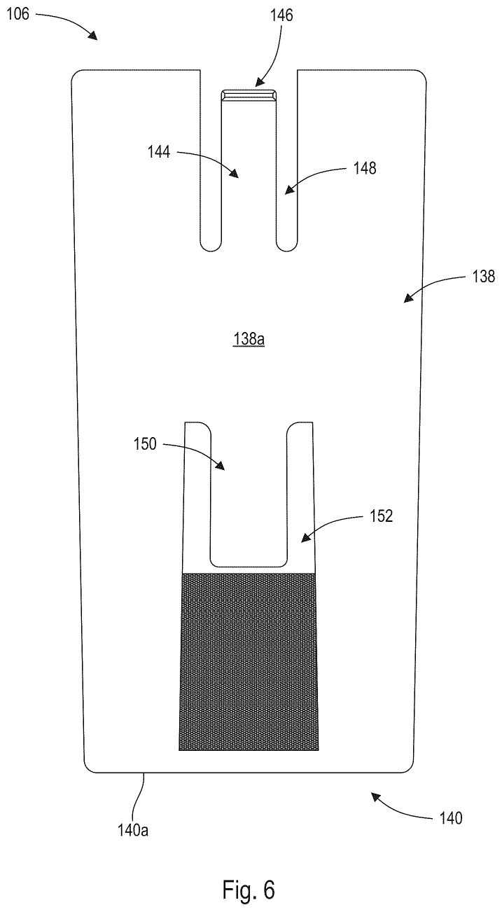

Referring now to FIGS. 6-8, the inner sleeve 106 is shown in accordance with an exemplary embodiment. The inner sleeve 106 includes a side wall 138 and a bottom wall 140. The side wall 138 includes an outer surface 138a and an opposed inner surface 138b. The bottom wall 140 includes an outer surface 140a and an opposed inner surface 140b. The side wall 138 extends substantially vertically from the bottom wall 140. The side wall 138 and the bottom wall 140 have generally circular cross-sectional profiles and are positioned concentrically relative to one another. The inner surface 138b of the side wall 138 and the inner surface 140b of the bottom wall 140 define an inner volume 142.

The side wall 138 defines a first and second deformable extension 144 that extend within a gap 148 of the side wall 138. The extensions 144 each include a hook 146. As will be discussed in further detail herein, the hooks 146 of the extensions 144 can be received by the openings 126 of the housing 102 to place which the lancet 100 in the undeployed position.

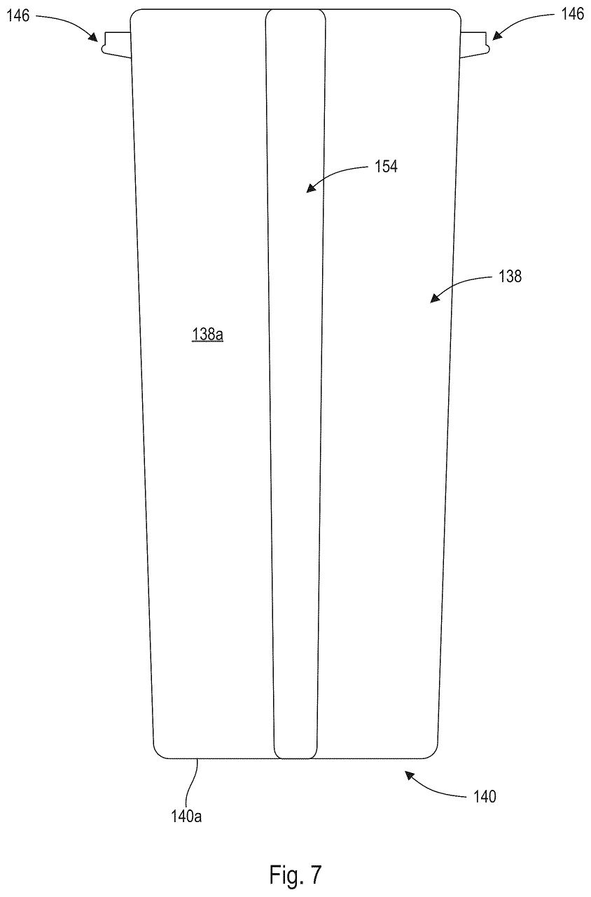

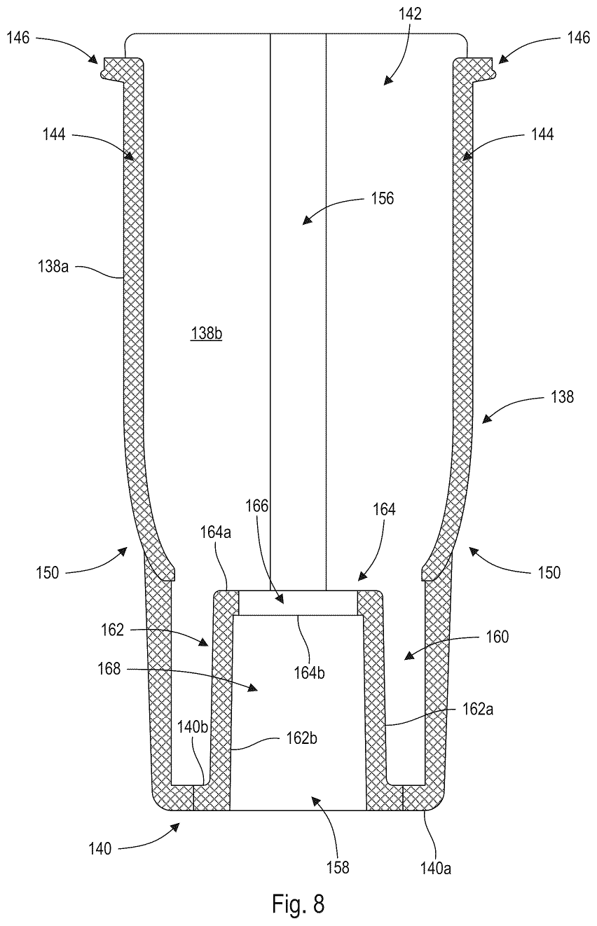

The side wall 138 further defines a first and second deformable tabs 150 that extend within an opening 152 of the side wall 138. The side wall 138 also includes a first and second protrusions 154 (only one of which is shown in FIG. 7) that extend substantially horizontally from and perpendicular to the outer surface 138a of the side wall 138. When the inner sleeve 106 is disposed within the housing 102, the protrusions 154 extend within the grooves 124 of the housing 102. When the inner sleeve 106 is moving between different positions (e.g., between an undeployed position to a deployed position), the grooves 124 and the protrusions 154 guide the vertical movement of the inner sleeve 106 while preventing a rotational or horizontal movement of the inner sleeve 106. The inner surface 138b of the side wall 138 further defines a first and second grooves 156 that are opposed from one another (only one of which is shown in FIG. 8). The grooves 156 extend substantially vertically from and perpendicular to the inner surface 140b of the bottom wall 140.

The inner sleeve 106 includes an opening 158 that extends through the bottom wall 140. That is, the opening 158 extends between the outer surface 140a and the inner surface 140b of the bottom wall 140. The inner sleeve 106 also includes a post 160 that is cylindrical in shape and is generally concentric with the opening 158. The post 160 includes a side wall 162 and a top wall 164. The side wall 162 extends substantially vertically from and perpendicular to the inner surface 140b of the bottom wall 140. The side wall 162 includes an outer surface 162a and an opposed inner surface 162b. The top wall 164 extends substantially horizontally between the side wall 162 and includes a top surface 164a and an opposed bottom surface 164b. The top wall 164 includes an opening 166 that extends through the top wall 164. That is, the opening 166 extends between the top surface 164a and the bottom surface 164b of the top wall 164. The inner surface 162b of the side wall 162 and the bottom surface 164b of the top wall 164 define an inner volume 168 of the post 160. As will be discussed in further detail herein, the needle(s) 110 extend through the opening 166 of the top wall 164, through the inner volume 168, and through the opening 158 of the bottom wall 140 when in the deployed position.

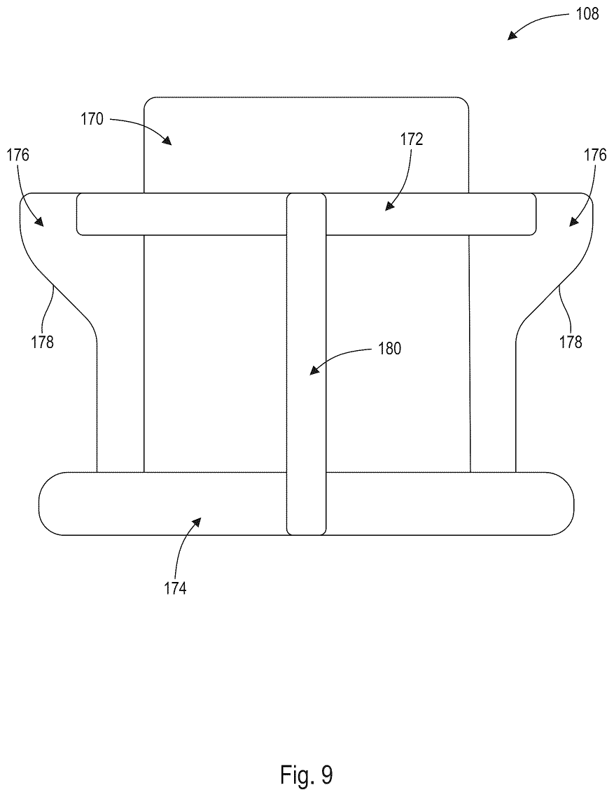

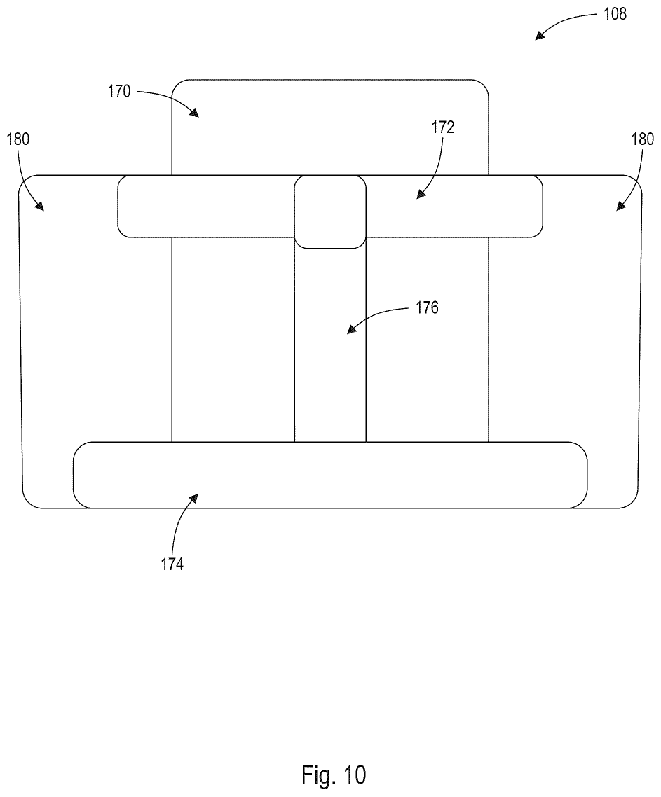

Referring now to FIGS. 9 and 10, the needle platform 108 is shown in accordance with an exemplary embodiment. The needle platform 108 includes a cylindrical body 170, an upper circular extension 172 and a lower circular extension 174. The upper circular extension 172 and the lower circular extension 174 extend substantially horizontally from and perpendicular to the cylindrical body 170. As will be discussed in further detail herein, the upper circular extension 172 serves as a platform for the injection spring 112. The needle platform 108 further includes a first and second curved extensions 176. The curved extensions 176 extend substantially horizontally from and perpendicular to the cylindrical body 170 and extend substantially vertically between the upper circular extension 172 and the lower circular extension 174. Each curved extension 176 includes an angled surface 178. As will be discussed in further detail herein, when the needle platform is in the undeployed position, the angled surfaces 178 contact the hooks 146 of the inner sleeve 106, which prevents the needle platform 108 from transitioning to the deployed position.

The needle platform 108 further includes first and second projections 180. The first and second projections 180 are generally rectangular in shape, extend substantially horizontally from and perpendicular to the cylindrical body 170, and extend substantially vertically between the upper circular extension 172 and the lower circular extension 174. When the needle platform 108 is disposed within the inner sleeve 106, the projections 180 extend into the grooves 156 which guides the vertical movement of the needle platform 108 while preventing a rotational or horizontal movement of the needle platform 108.

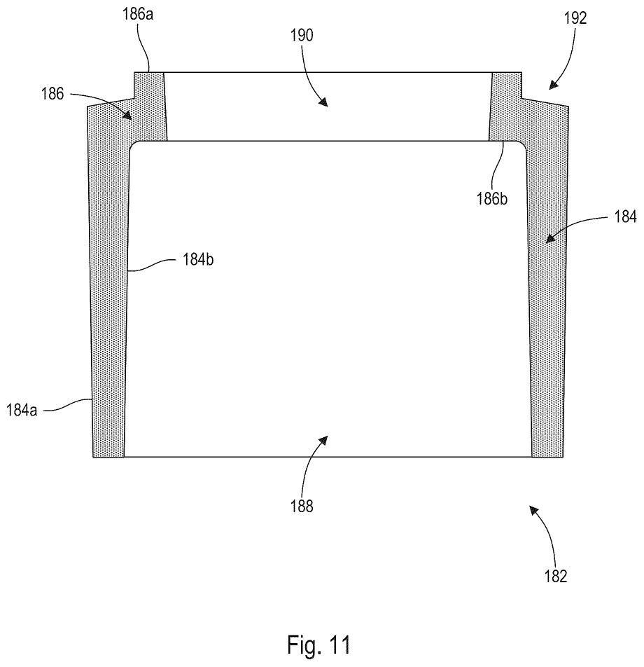

As depicted in FIG. 3, the lancet 100 further includes a spring sleeve 182. With particular reference to FIG. 11, the spring sleeve 182 is shown in accordance with an exemplary embodiment. The spring sleeve 182 is cylindrical in shape and includes a side wall 184 and a top wall 186. The side wall 184 includes an outer surface 184a and an opposed inner surface 184b and the top wall 186 includes a top surface 186a and an opposed bottom surface 186b. The inner surface 184b of the side wall 184 and the bottom surface 186b of the top wall 186 define an inner volume 188 of the spring sleeve 182. The side wall 184 and the top wall 186 are shaped and dimensioned such that the spring sleeve 182 retains the retraction spring 114 within the inner volume 188. The top wall 186 includes an opening 190 that extends therethrough. That is, the opening 190 extends between the top surface 186a and the bottom surface 186b of the top wall 186. The opening 190 of the top wall 186 is in communication with the inner volume 188. Since the inner volume 188 is open, the needle(s) 110 may extend through spring sleeve 182 via the opening 190. The top surface 184a defines a ledge 192 that extends circumferentially around the spring sleeve 182. As will be discussed in further detail herein, when the lancet 100 is in the undeployed position, the tabs 150 contact the ledge 192.

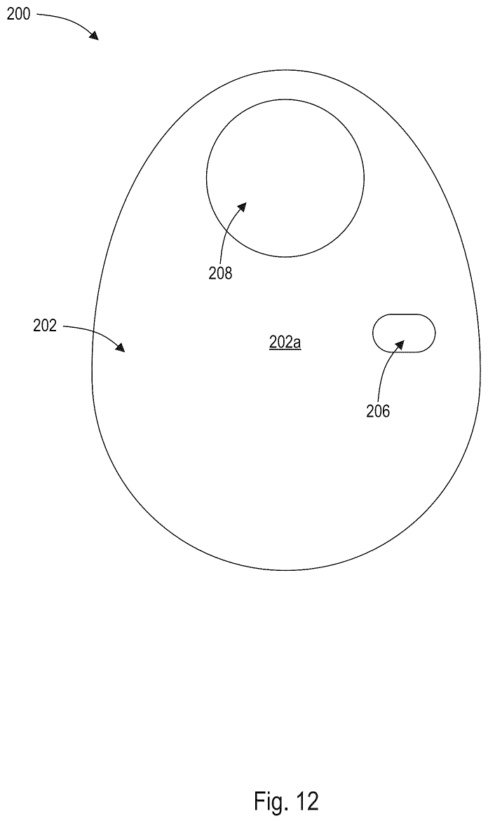

Referring now to FIGS. 12-17, the cartridge 12 is shown in accordance with an exemplary embodiment. The cartridge 12 is configured to attach to the skin of a subject and includes an adhesive layer (not shown) disposed on a bottom surface of the cartridge 12. After use, the cartridge 12 may be removed by pulling the cartridge 12 off the skin of the subject. The cartridge 12 includes a cover 200 and a base 300 that can couple to the cover 200. In one embodiment, the cover 200 and the base 300 are formed as two separate components that are removably coupled to one another (e.g., via a snap fitting). In another embodiment, the cover 200 and the base 300 form an integral unitary cartridge 12. The cover 200 and the base 300 may be coupled to one another via an adhesive, laser welding, etc.

The cartridge 12 may be formed using a variety of suitable materials including, but not limited to, polymeric materials (e.g., polyolefins, polyethylene terephthalate (PET), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, photocross linkable polymers, etc.). In some embodiments, some of the cover 200 may be formed of poly(dimethylsiloxane) (PDMS) to allow visibility of components disposed within the cartridge 12.

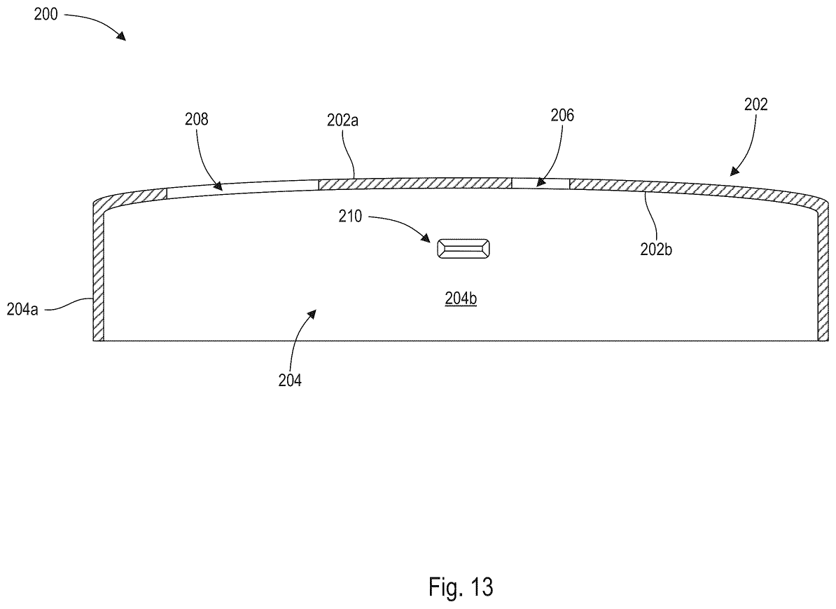

Referring now to FIGS. 12 and 13, the cover 200 is shown in accordance with an exemplary embodiment. The cover 200 includes a top wall 202 with an outer surface 202a and an opposed inner surface 202b. The cover 200 also includes a side wall 204 with an outer surface 204a and an opposed inner surface 204b. The top wall 202 extends substantially longitudinally from and perpendicular to the side wall 204. The side wall 204 extends substantially vertically from and perpendicular to the top wall 202.

The cover 200 includes a viewing aperture 206 that extends through the top wall 202. That is, the viewing aperture 206 extends between the outer surface 202a and the inner surface 202b of the top wall 202. In various embodiments, the viewing aperture 206 is covered by a transparent material, which allows a user of the dermal patch system to view components disposed within the cartridge 12. The top wall 202 further includes a lancet aperture 208 that extends through the top wall 202. That is, the lancet aperture 208 extends between the outer surface 202a and the inner surface 202b of the top wall 202. The lancet aperture 208 is generally shaped and dimensioned to accept a portion of the lancet 100, e.g., the aperture can be substantially circular. As will be discussed in further detail herein, a portion of the lancet 100 extends through the lancet aperture 208 to couple to the base 300.

The cover 200 further includes a plurality of locking members 210 that extend substantially horizontally from and perpendicular to the inner surface 204a of the side wall 204. As will be discussed in further detail herein, the locking members 210 aid in coupling the cover 200 to the base 300.

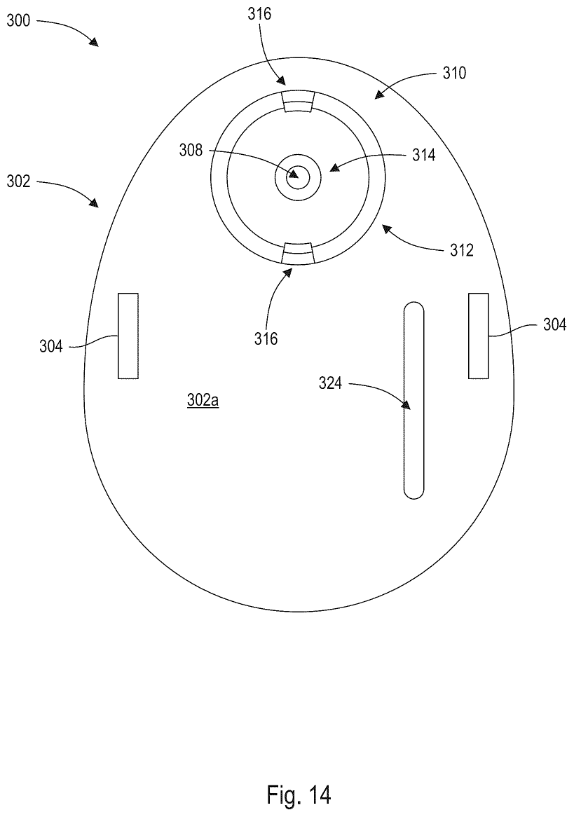

Referring now to FIGS. 14-17, the base 300 is shown in accordance with an exemplary embodiment. The base 300 includes a bottom wall 302 that includes a top surface 302a and opposed bottom surface 302b. The bottom wall 302 has a similar shape and dimension as the side wall 204 of the cover 200 such that the cover 200 and the base 300 are flush with one another when the cover 200 is coupled to the base 300. Furthermore, when the cover 200 is coupled to the base 300, the side wall 204 contacts the bottom wall 302.

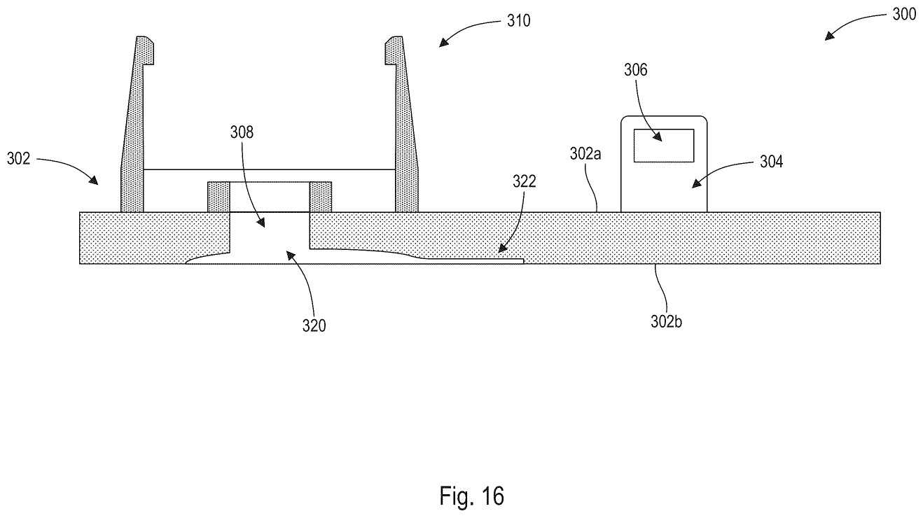

The base 300 includes a plurality of extensions 304 that extend substantially vertically from and perpendicular to the top surface 302a of the bottom wall 302. Each of the extensions 304 define a gap 306 (FIG. 16). The gap 306, and therefore the extensions 304, are shaped and dimensioned to accept a locking member 210. The locking members 210 extend through the gaps 306 to couple the cover 200 to the base 300.

The base 300 further includes a needle aperture 308 that is generally circular in shape. The needle aperture 308 extends through the bottom wall 302. That is, the needle aperture 308 extends between the top surface 302a and the bottom surface 302b of the bottom wall 302. As will be discussed in further detail herein, when the cover 200 is coupled to the base 300 and when the cartridge 12 is adhered to a subject, the needle aperture 308 allows the needle(s) 110 of the lancet 100 to extend through the bottom wall 302 to puncture the subject's skin.

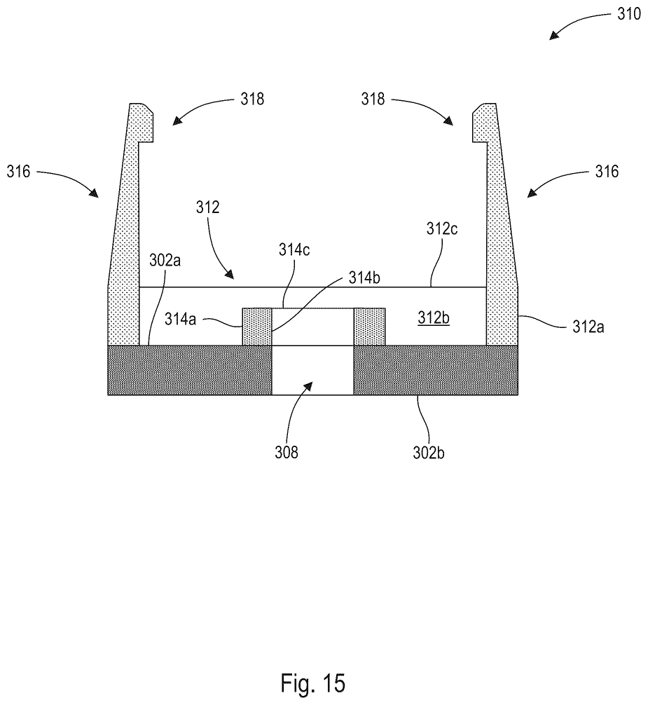

The base 300 also includes lancet receiving element 310 that is shaped and dimensioned to accept a distal end of the lancet 100. With particular reference to FIG. 15, the lancet receiving element 310 includes an outer circular projection 312 and an inner circular projection 314 (see FIG. 14), each extending substantially vertically from and perpendicular to the top surface 302a of the bottom wall 302. The outer circular projection 312 includes an outer surface 312a, an opposed inner surface 312b, and a top surface 312c that extends between the outer surface 312a and the inner surface 312b. The top surface 312c extends substantially perpendicular to and horizontally between the outer surface 312a and the inner surface 312b. The outer surface 312a and the inner surface 312b extend substantially vertically from and perpendicular to the top surface 302a of the bottom wall 302. The outer circular projection 312 is shaped to accept the lancet 100.

The inner circular projection 314 is disposed around the needle aperture 308 and includes an outer surface 314a, an opposed inner surface 314b, and a top surface 314c that extends between the outer surface 314a and the inner surface 314b. The top surface 314c extends substantially perpendicular to and horizontally between the outer surface 314a and the inner surface 314b. The outer surface 314a and the inner surface 314b extend substantially vertically from and perpendicular to the top surface 302a of the bottom wall 302. The outer circular projection 312 and the inner circular projection 314 circular projection are substantially concentric with one another.

As will be discussed in further detail herein, when the lancet 100 is engaged with the cartridge 12, the top surface 314c of the inner circular projection 314 contacts a portion of the lancet 100 which causes the lancet 100 to transition the needle(s) 110 from the undeployed position to the deployed position.

Withe continued reference to FIG. 15, the lancet receiving element 310 further includes a plurality of locking members 316 that extend substantially vertically from and perpendicular to the top surface 312c of the outer circular projection 312. While FIGS. 14 and 15 depict the lancet receiving element 310 as including two locking members 316, it is understood that, in other embodiments, the lancet receiving element 310 may include more or less locking members 316 (e.g., 1, 3, 5, etc.). Each locking member 316 includes a hook 318 that extends inwardly from a top of a locking member 316 towards the inner circular projection 314. As will be discussed in further detail herein, the hooks 318 of the locking members 316 couple to the lancet 100 to retain the lancet 100 within the base 300.

With particular reference to FIG. 16, the base 300 includes a physiological sample well 320 and a physiological sample channel 322 that extends from and is in open communication with the physiological sample well 320. As will be discussed in further detail herein, the physiological sample channel 322 is a fluidic channel that is configured to carry a physiological sample extracted from a subject. The physiological sample well 320 and a portion of the physiological sample channel 322 are open with respect to the bottom surface 302b of the bottom wall 302. Stated another way, the physiological sample well 320 and a portion of the physiological sample channel 322 do not include a bottom surface. The physiological sample well 320 is in open communication with the needle aperture 310. As will be discussed in further detail herein, when drawing a physiological sample, the needle(s) 110 of the lancet 100 extends through the needle aperture 308 and through the physiological sample well 320 to pierce the skin of the subject. The physiological sample channel 322 extends from the physiological sample channel 322 and through the bottom wall 302 of the base 300.

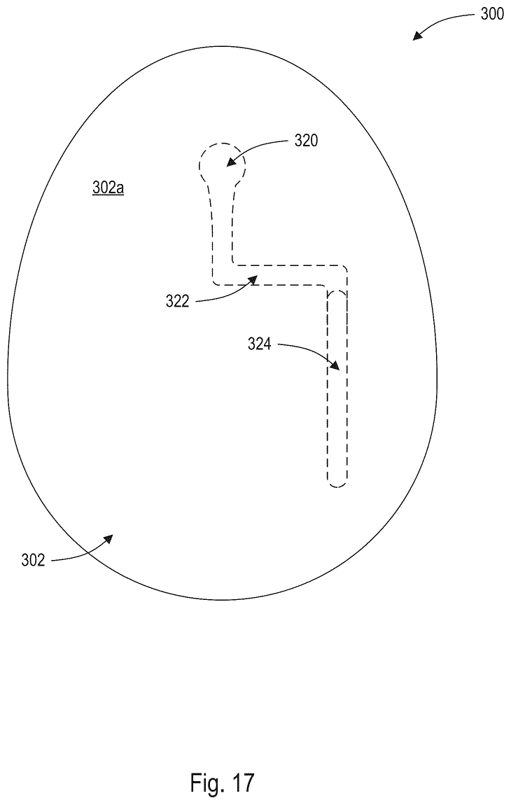

The bottom wall 302 of the base 300 further includes a sample collection reservoir 324 (FIG. 17) that is configured to receive and store a drawn physiological sample. The sample collection reservoir 324 is in open communication with the physiological sample channel 322. Accordingly, the physiological sample channel 322 carries a drawn physiological sample from the physiological sample well 320 to the sample collection reservoir 324. The sample collection reservoir 324 is disposed vertically below the viewing aperture 206. In one embodiment, the collection reservoir 324 is covered by a transparent material which allows a user of the dermal patch system 10 to view the sample collection reservoir 324 via the viewing aperture 206. In some embodiments, a sample collection pad (e.g., a CF12 collection pad) is disposed within the sample collection reservoir 324 and absorbs the drawn physiological sample.

The base 300 includes an adhesive layer (not shown) disposed on the bottom surface 302b of the bottom wall 302. The cartridge 12 can be attached to the skin of a subject via the adhesive layer. The cartridge 12 may be attached anywhere on the subject's skin that is capable of supporting the cartridge 12 (e.g., on a leg, arm, etc. of the subject). The adhesive layer includes an opening that surrounds the physiological sample well 320 and through which the needle(s) 110 can extend to puncture the subject's skin. The adhesive layer covers and therefore seals the open portion of the physiological sample channel.

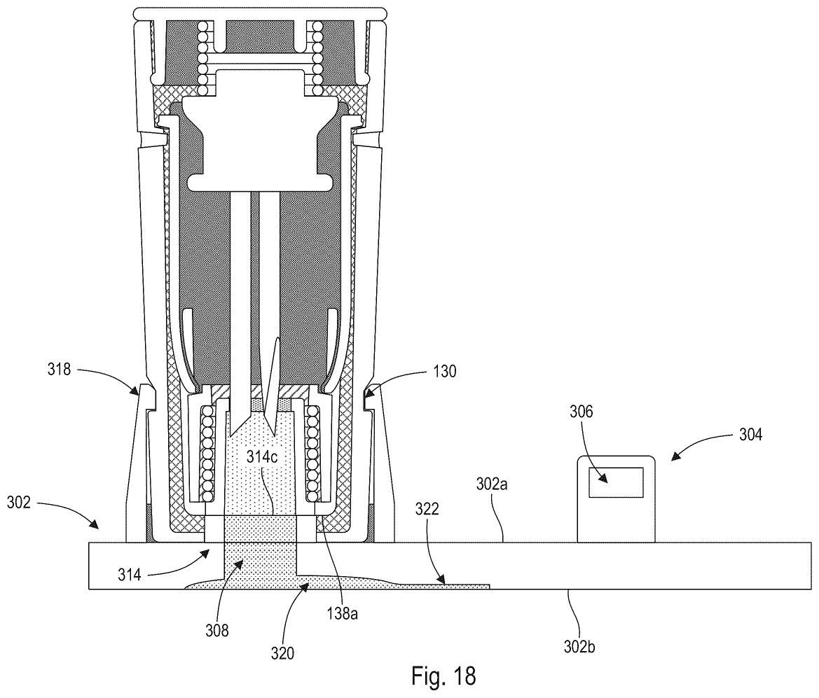

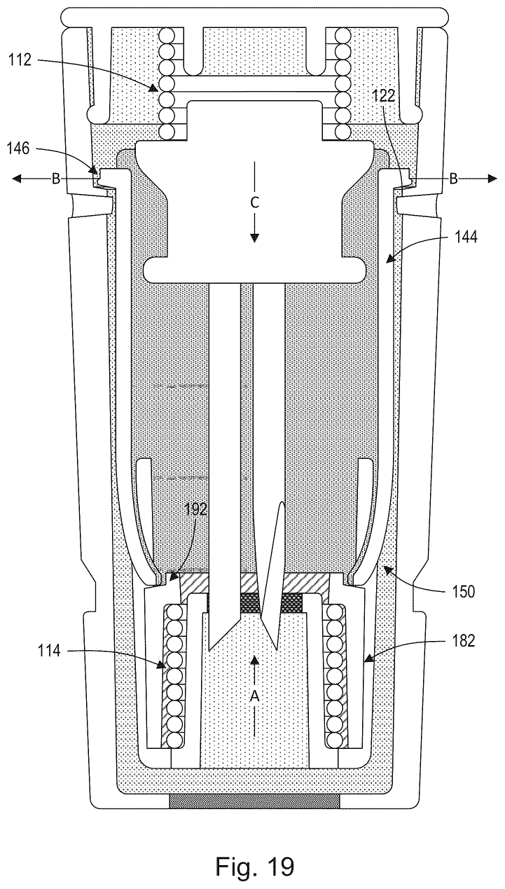

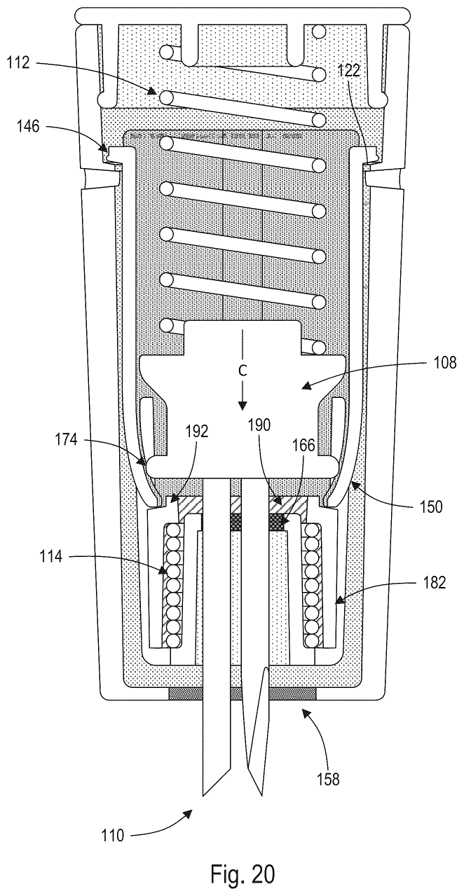

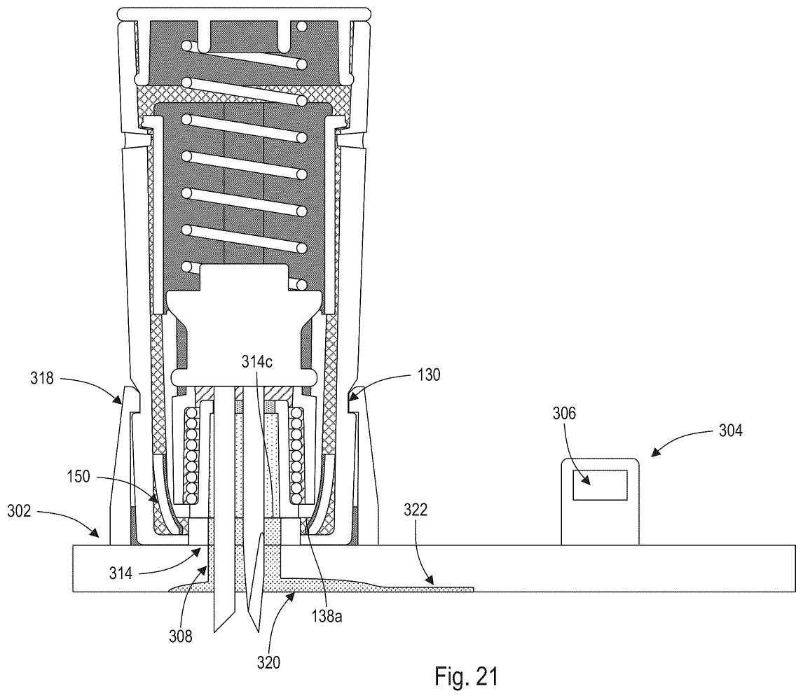

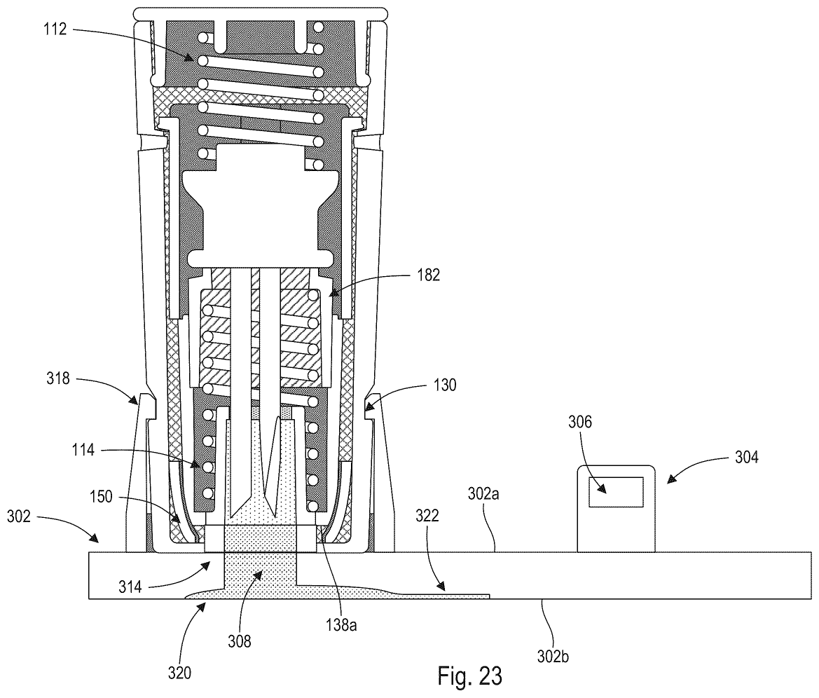

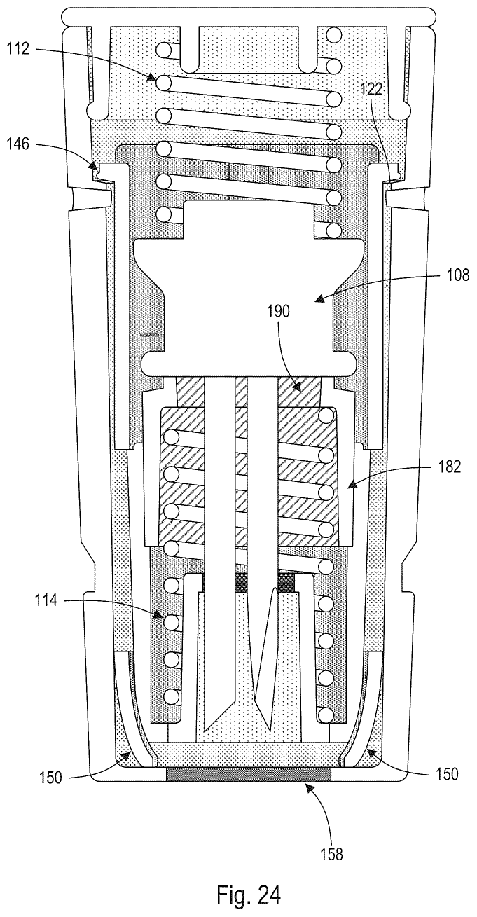

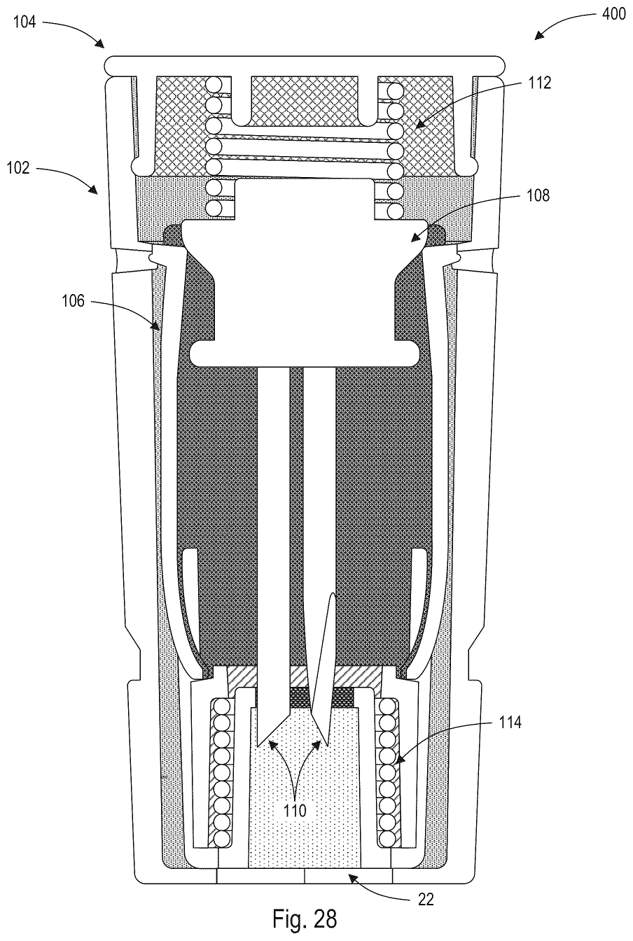

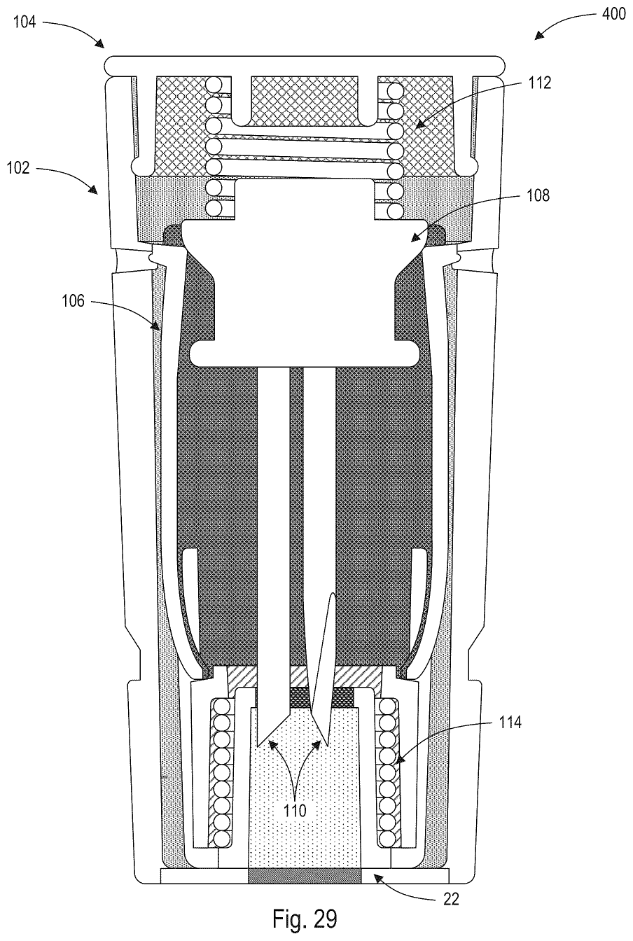

The lancet 100 is moveable between an undeployed position (FIG. 18 and FIG. 19), a deployed position (FIGS. 20, 21 and 22), and a retracted position (FIGS. 23 and 24). A fully retracted position is shown in FIG. 28 and FIG. 29.

In the undeployed position, the hooks 146 (see FIG. 8) of the inner sleeve 106 extend through the openings 126 of the housing 102, which retains the lancet 100 in the undeployed position and prevents the lancet 100 from moving to the deployed position. In this position, the extensions 144 are compressed inward toward the center of the lancet 100 which allows the needle platform 108 to rest upon the inner sleeve 106 thereby preventing the needle platform from moving vertically downward and into the deployed position. Specifically, the angled surface 178 of the extensions 176 of the needle platform 108 contacts rests upon the extensions 144.

Furthermore, the injection spring 112 extends around the body 170 of the needle platform 108 and the outer surface 136a of the inner cylinder 136. The injection spring 112 extends vertically between the inner surface 132b of the top wall 132 of the cap 104 and the upper circular extension 172 of the needle platform 108. In the undeployed position, the injection spring 112 is compressed between the top wall 132 of the cap 104 and the upper circular extension 172 of the needle platform 108. The retraction spring 114 extends around the post 160 of the inner sleeve 106. The retraction spring 114 extends vertically between the bottom wall 140 of the inner sleeve 106 and the top wall 186 of the spring sleeve 182. That is, the retraction spring 114 extends vertically between the inner surface 140b of the bottom wall 140 of the inner sleeve 106 and the inner surface 186b of the top wall 186 of the spring sleeve 182. When the lancet 100 is in the undeployed position, the retraction spring 114 is compressed between the bottom wall 140 of the inner sleeve 106 and the top wall 186 of the spring sleeve 182. Furthermore, in the undeployed position, the deformable tabs 150 contact the ledge 192 of the spring sleeve 182 so as to prevent the retraction spring from expanding and moving the spring sleeve 182.

The lancet 100 may be coupled to the cartridge 12 by pushing the lancet 100 through the lancet aperture 208 and into the base 300. This engagement causes the needle(s) 110 of the lancet 100 to automatically move from an undeployed position to a deployed position and automatically from the deployed position to a retracted position. Furthermore, when the lancet 100 engages with the base 300, the hooks 318 of the locking members 316 couple to the groove 130 of the housing 102 via a snap fitting. This coupling causes the generation of an audible clicking sound that indicates to a user that the lancet 100 was correctly inserted into the cartridge 12.

As depicted in FIGS. 18, 21, and 23, when the lancet 100 is coupled to the base 300 the inner circular projection 314 of the lancet receiving element 310 extends through the aperture 128 of the housing 102 and contacts the bottom wall 140 of the inner sleeve 106. Specifically, the top surface 314c of the inner circular projection 314 contacts the outer surface 140a of bottom wall 140 which forces the inner sleeve 106 to move vertically upward in the direction of arrow A within the housing 102. As previously discussed herein, grooves 124 of the housing 102 and the protrusions 154 of the inner sleeve 106 guide the vertical movement of the inner sleeve 106. The vertical movement of the inner sleeve 106 causes the hooks 146 to disengage from the openings 126 and causes the extensions 144 to extend vertically above and rest upon the ledge 122. In this position, the extensions 144 decompress and expand outwardly in the direction of arrow B.

When the extensions 144 expand, the needle platform 108 no longer rests upon the extensions 144 and hence can move vertically downward in the direction of arrow C (see FIG. 20). As the needle platform 108 moves downward, the injection spring 112 is allowed to expand and the force applied by the injection spring 112 causes the needle platform 108 (and therefore the needle(s) 110) to travel with at a rate that is sufficient to cause the needle(s) 110 to puncture the skin of a subject wearing the dermal patch system 10. The expansion of the injection spring 112 causes the needle(s) 110 to move and extend through the spring sleeve 182 via the opening 190, through the post 160 via the opening 166, through the opening 158 of the inner sleeve 106 and exit the housing 102 via the aperture 128 and pass through the base 300 of the cartridge 12 via the needle aperture 308. Stated another way, the injection spring 112 moves the needle(s) 110 to the deployed position.

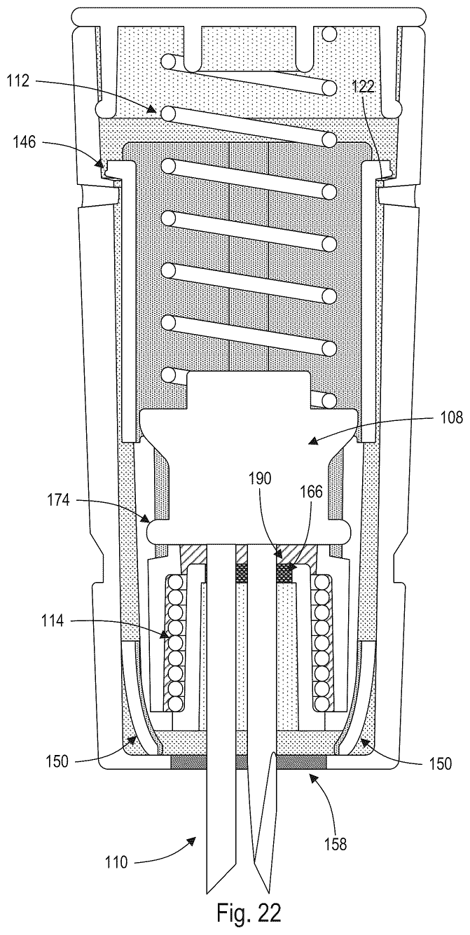

As previously discussed herein the grooves 156 of the inner sleeve 106 and the projections 180 of the needle platform 108 guide the vertical movement of the needle platform 108. As the needle platform moves in the direction of arrow B, the lower circular extension 174 contacts the deformable tabs 150 (FIG. 20). This contact causes the tabs 150 to deflect away from the spring sleeve 182 of the lancet 100 or causes the tabs 150 to break. As depicted in FIG. 22, when the tabs 150 break, the broken piece(s) of the tabs 150 fall into a void between the inner sleeve 106 and the housing 102. In the deployed position, the needle(s) 110 extend beyond the housing 102 and the lower circular extension 174 contacts the top wall 186 of the spring sleeve 182.

When the tabs 150 deflect away from the spring sleeve 182 or break, the tabs 150 no longer contact the ledge 192 of the spring sleeve 182. As a result, the retraction spring 114 is allowed to expand. The expansion of the retraction spring 114 moves the spring sleeve 182 vertically upward in the direction of arrow D. If the tabs 150 deflect and do not break, the spring sleeve 182 prevents the tabs 150 from contacting the retraction spring 114 as the retraction spring 114 expands. The spring sleeve 182 moves the needle platform 108 in the direction of arrow D to place the lancet 100 in the retracted position. That is, after moving to the deployed position, the retraction spring 114 causes the needle(s) 110 to retract back into the inner volume 142 of the inner sleeve 106 via the needle aperture 308 of the base 300, the aperture 128 of the housing 102, and the opening 158 of the inner sleeve 106. That is, after penetrating the skin of a subject, the retraction spring 114 causes the needle(s) 110 to automatically retract back into the housing of the lancet 100 thereby placing the lancet 100 in the retracted position.

In this embodiment, the three needles 110 have substantially the same length, and by simultaneously moving the three needles 110 to the deployed position, the lancet 100 punctures the skin of the subject at three locations at the same time, though in other embodiments, two or all of the three needles 110 may have different lengths.

In some embodiments, the use of three needles 110 results in placing a portion of the skin under tension, which can in turn facilitate drawing blood through on or more punctures caused by the needles. In particular, as noted above, it has been discovered that the use of three needles 110 can advantageously allow a larger volume of blood to be drawn compared to the use of one, two, or a greater number of needles 110. The invention is not limited to three needles. One, two, three or more needles can be used. Without being limited to any particular theory, in some embodiments, in addition to placing a skin portion under tension, the use of three needles 110 may also improve the likelihood that at least one of the needles would cut through a capillary to extract a blood volume from the subject. This is because when the skin is tensioned, one of the needles can penetrate the skin at a different depth. As a result, it is possible to have a higher volume of blood flow out of one of the punctures relative to the other two. Also, when three needles are used, one of the needles can be used to tent the skin to improve the likelihood of puncturing the skin. Each of the needles 110 can has a tapered tip that points outward (away from the center of the lancet) to maximize a distance between puncture locations on the subject's skin. Having a greater distance between puncture points can increase the likelihood that a capillary is targeted, thereby increasing the probability of bleeding. Furthermore, the three needles 110 may be positioned such that the needle tips form an equilateral triangle and are therefore equally space from one another.

It is understood that the lancet 100 may be used with any cartridge of a dermal patch system that is configured to engage with the lancet 100. That is, the lancet 100 may be used with any cartridge that includes an extension that causes the lancet 100 to transition from the undeployed position to the deployed position.

After the needles 110 retract, a physiological sample pools within the physiological sample well 320 of the base 300. Due to gravity, wicking, and capillary action, the physiological sample travels from the physical sample well 320 to the sample collection reservoir 324 via the physiological sample channel 322. A user can view the sample collection reservoir 324 via the viewing aperture 206 to ensure the physiological sample has traveled to the sample collection reservoir 324. After drawing the physiological sample, the cartridge 12 may be removed from the skin of the subject and sent to a laboratory for analysis. A medical professional may then extract the physiological sample and analyze the sample.

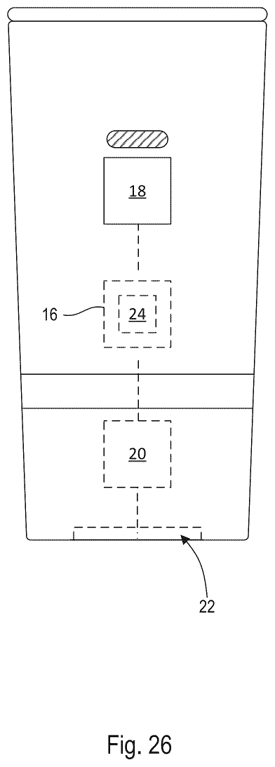

As shown in FIG. 26, the lancet 400 can include a sensor 18 that can create a digital image of the fingerprint of the user of the lancet and electronics 16 that process the digital image. The sensor 18 can be a fingerprint sensor, or any type of biometric sensor that can identify the user of the lancet. For example, a finger image sensor (FIS), or a contact image sensor (CIS), such as the M116-A8CIU sensor or the M116-A6CIP sensor made by CMOS Sensor Inc. in Cupertino, CA, can be used.

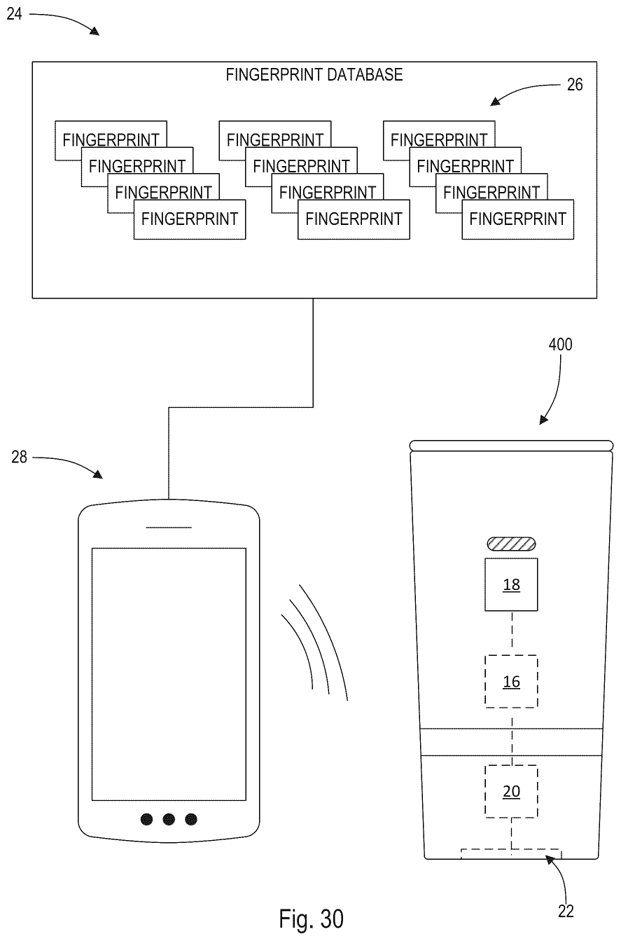

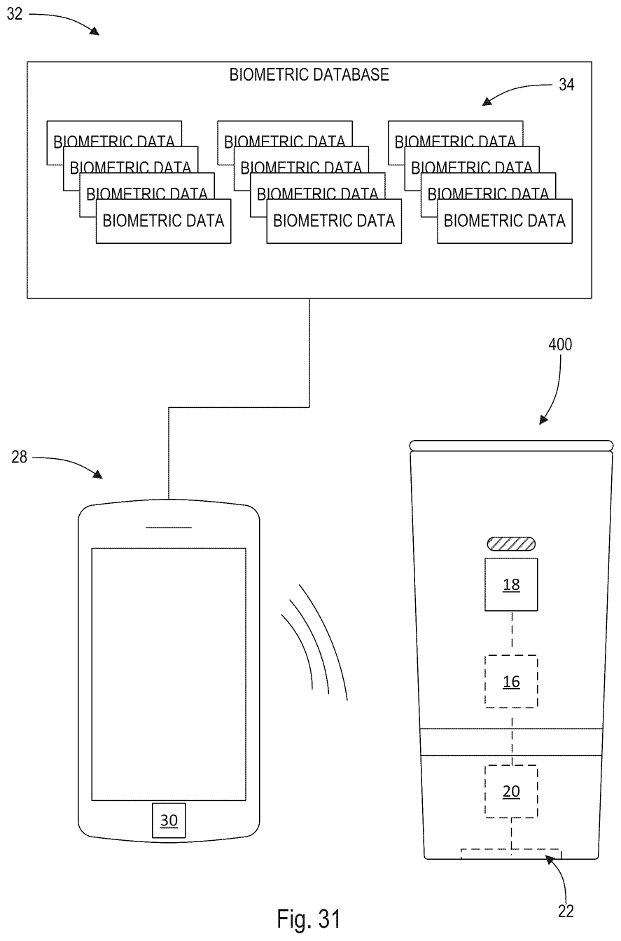

In another embodiment (FIG. 30), a first computer system 28 is connected to a fingerprint database 24 that includes data for a plurality of fingerprints 26, each associated with a different subject. In this embodiment, before drawing a physiological sample from a subject, the computer system 28 receives via Bluetooth, or other wireless communication method, the fingerprint image output by the sensor 18 on the lancet 400. As described above, the sensor 18 can scan the fingerprint of the finger that is applied to the lancet 400 and produce a digital image of the user's fingerprint. The computer system 28 then can identify the subject by comparing the digital image of the user's fingerprint with the fingerprint data 26. The identity of the subject can then be associated with the QR code on the cartridge 12. While the above describes a system that identifies a subject based on a fingerprint, the system may identify a subject from which a physiological sample has been drawn based on any database that includes a subject's identity, such as the biometric database 32 shown in FIG. 31.

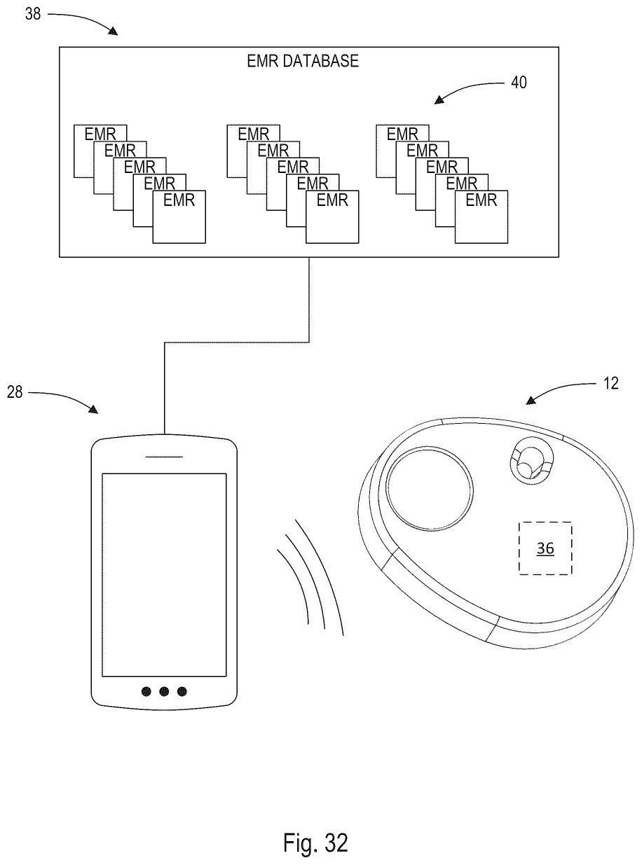

In another embodiment (FIG. 32), a first computer system 28 is connected to an electronic medical record (“EMR”) database 38 that includes a plurality of EMR's 40 each associated with a different subject. In this embodiment, after drawing a physiological sample from a subject, the computer system 28 scans the QR code 36 on the cartridge 12 and sends the cartridge 12 to a laboratory for further analysis. In response to scanning the QR code 36, computer system 28 associates the QR code 36 with an EMR 40 that corresponds to the subject from which the physiological sample was drawn. In some embodiments, the computer system 28 may update the associated EMR 40 to indicate a physiological has been drawn automatically or based on a user input. While the above describes a system for identifying a subject from which a physiological sample has been drawn by matching QR codes in an EMR database, the system may identify a subject from which a physiological sample has been drawn by matching the physiological sample to any database that includes a subject's identity, such as the fingerprint database shown in FIG. 30 and the biometric database shown in FIG. 31.

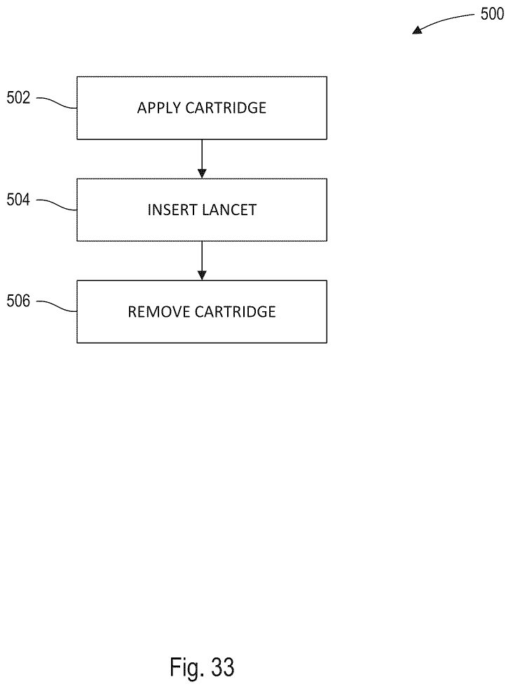

Referring now to FIG. 33, a method 500 for obtaining a physiological sample from a subject is disclosed herein.

At 502, a cartridge 12 of the dermal patch system 10 is affixed to the skin of a subject as previously discussed herein.

At 504, the lancet 100 is inserted into the cartridge 12 to draw a physiological sample (e.g., a blood sample, a sample of interstitial fluid, etc.) from the subject as previously discussed herein.

At 506, the user removes the cartridge 12 from the skin of the subject as previously discussed herein.

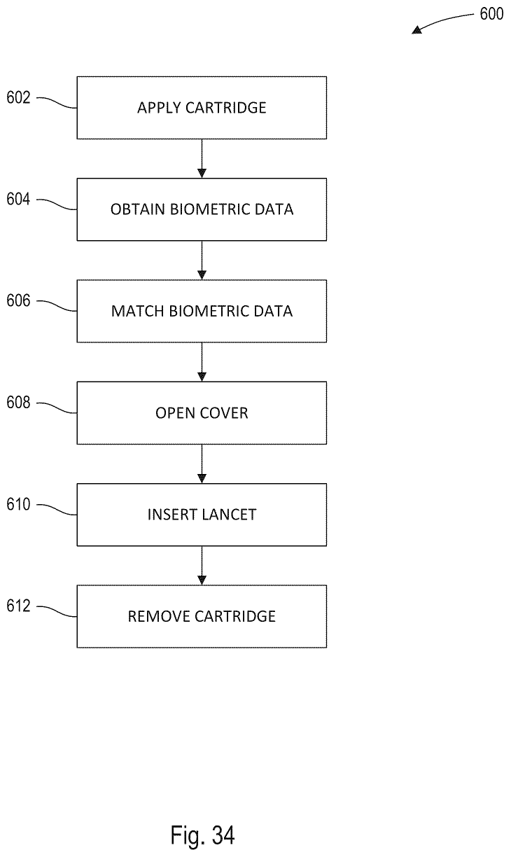

Referring now to FIG. 34, a method 600 for obtaining a physiological sample from a subject is disclosed herein. At 602, a user attaches the cartridge 12 of the dermal patch system 10 to the skin of a subject at a suitable location (e.g., arm, leg, etc.) as previously discussed herein. At 604, a biometric sensor obtains biometric data from the user of the dermal patch system 10 and sends a signal indicative of the biometric data to the computer system 28 as previously discussed herein. At 606, in response to receiving the signal indicative of the biometric data, the computer system determines whether the biometric data matches the biometric data stored in the memory of the computer system. At 608, the cover 200 of the cartridge 12 is opened to allow access of the lancet into the cartridge 12. At 610, the lancet 100 is inserted in the cartridge 12 to engage with the cartridge 12 to draw the physiological sample. At 612, the cartridge 12 is removed from the skin of the subject as previously discussed herein. The cartridge 12 can then be sent to a medical professional to analyze the drawn physiological sample as previously discussed herein.

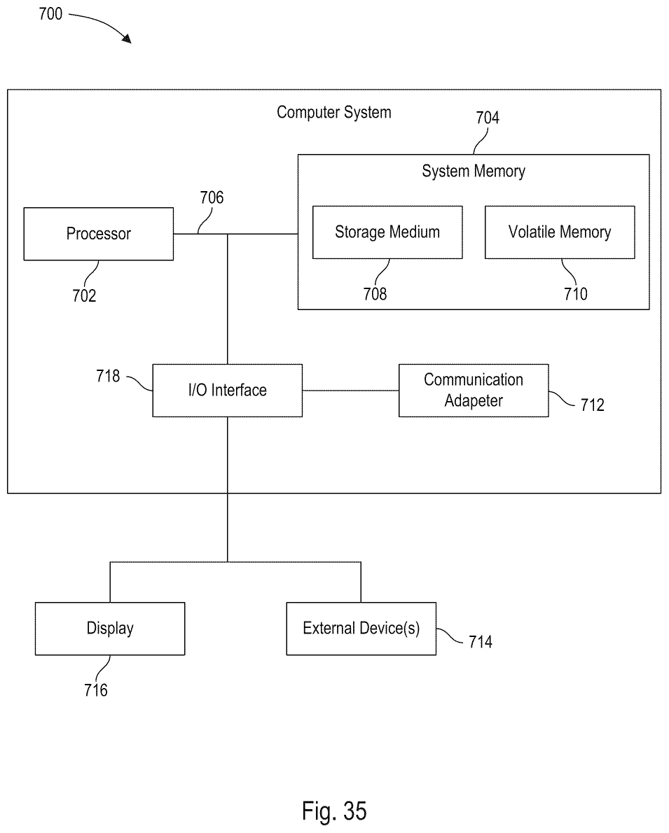

Referring now to FIG. 35, a computer system 700 is shown in accordance with an exemplary embodiment. The computer system 700 may serve as any computer system disclosed herein (e.g., the computer system 28 in FIGS. 30, 31 and 32). As used herein a computer system (or device) is any system/device capable of receiving, processing, and/or sending data. Computer systems include, but are not limited to, microprocessor-based systems, personal computers, servers, hand-held computing devices, tablets, smartphones, multiprocessor-based systems, mainframe computer systems, virtual reality (“VR”) headsets and the like.

As shown in FIG. 35, the computer system 700 includes one or more processors or processing units 702, a system memory 704, and a bus 706 that couples the various components of the computer system 700 including the system memory 704 to the processor 702. The system memory 704 includes a computer readable storage medium 708 and volatile memory 710 (e.g., Random Access Memory, cache, etc.). As used herein, a computer readable storage medium includes any media that is capable of storing computer readable; program instructions and is accessible by a processor. The computer readable storage medium 708 can include non-volatile and non-transitory storage media (e.g., flash memory, read only memory (ROM), hard disk drives, etc.). Computer program instructions as described herein include program modules (e.g., routines, programs, objects, components, logic, data structures, etc.) that are executable by a processor. Furthermore, computer readable program instructions, when executed by a processor, can direct a computer system to function in a particular manner such that a computer readable storage medium comprises an article of manufacture. Specifically, the computer readable program instructions when executed by a processor can create a means for carrying out at least a portion of the steps of the methods disclosed herein.

The bus 706 may be one or more of any type of bus structure capable of transmitting data between components of the computer system 700 (e.g., a memory bus, a memory controller, a peripheral bus, an accelerated graphics port, etc.).

The computer system 700 may further include a communication adapter 712 which allows the computer system 700 to communicate with one or more other computer systems/devices via one or more communication protocols (e.g., Wi-Fi, BTLE, etc.) and in some embodiments may allow the computer system 700 to communicate with one or more other computer systems/devices over one or more networks (e.g., a local area network (LAN), a wide area network (WAN), a public network (the Internet), etc.).

In some embodiments, the computer system 700 may be connected to one or more external devices 714 and a display 716. As used herein, an external device includes any device that allows a user to interact with a computer system (e.g., mouse, keyboard, touch screen, etc.). An external device 714 and the display 716 may be in communication with the processor 702 and the system memory 904 via an Input/Output (I/O) interface 718.

The display 716 may display a graphical user interface (GUI) that may include a plurality of selectable icons and/or editable fields. A user may use an external device 714 (e.g., a mouse) to select one or more icons and/or edit one or more editable fields. Selecting an icon and/or editing a field may cause the processor 702 to execute computer readable program instructions stored in the computer readable storage medium 708. In one example, a user may use an external device 714 to interact with the computer system 700 and cause the processor 702 to execute computer readable program instructions relating to at least a portion of the steps of the methods disclosed herein.

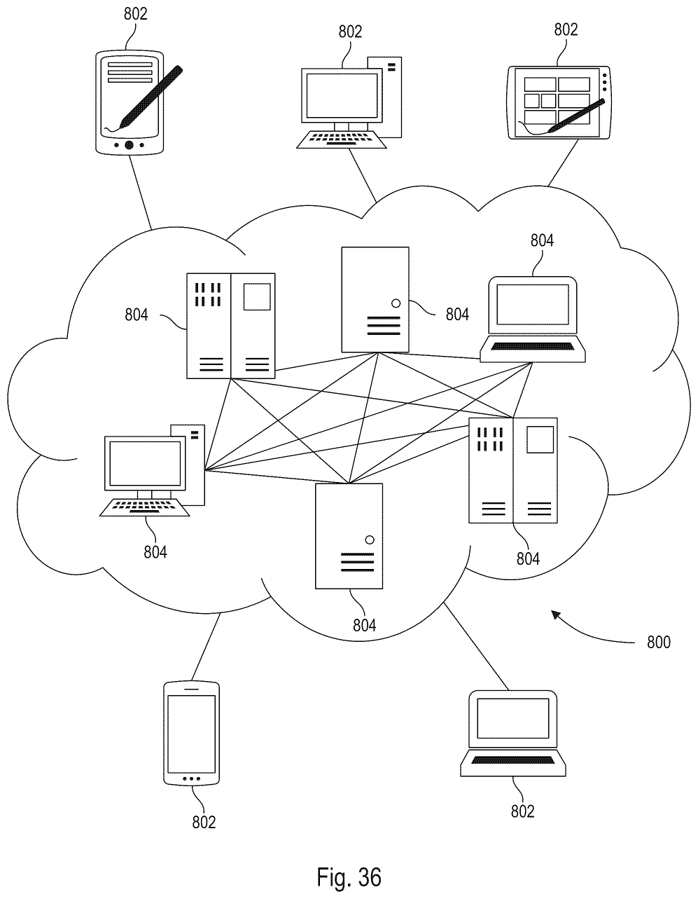

Referring now to FIG. 36, a cloud computing environment 800 is depicted in accordance with an exemplary embodiment. The cloud computing environment 800 is connected to one or more user computer systems 802 and provides access to shared computer resources (e.g., storage, memory, applications, virtual machines, etc.) to the user computer systems 802. As depicted in FIG. 36, the cloud computing environment includes one or more interconnected nodes 804. Each node 804 may be a computer system or device local processing and storage capabilities. The nodes 804 may be grouped and in communication with one another via one or more networks. This allows the cloud computing environment 800 to offer software services to the one or more computer services to the one or more user computer systems 802 and as such, a user computer system 802 does not need to maintain resources locally.

In one embodiment, a node 804 includes computer readable program instructions for carrying out various steps of various methods disclosed herein. In these embodiments, a user of a user computer system 802 that is connected to the cloud computing environment may cause a node 804 to execute the computer readable program instructions to carry out various steps of various methods disclosed herein.

As previously discussed, the above may be implemented by way of computer readable instructions, encoded or embedded on computer readable storage medium (which excludes transitory medium), which, when executed by a processor(s), cause the processor(s) to carry out the methods of the present disclosure.

While various embodiments have been illustrated and described in detail in the drawings and foregoing description, such illustration and description are to be considered illustrative or exemplary and not restrictive; embodiments of the present disclosure are not limited to the disclosed embodiments. Other variations to the disclosed embodiments can be understood and effected by those skilled in the art in practicing embodiments of the present disclosure, from a study of the drawings, the disclosure, and the appended claims.

In the claims, the word “comprising” does not exclude other elements or steps, and the indefinite article “a” or “an” does not exclude a plurality. A single processor or other processing unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

What is claimed is:

1. A dermal patch system comprising:
a lancet including one or more needles, and
a cartridge configured to attach to the skin of a subject,
wherein the lancet is configured automatically move the one or more needles from an undeployed position to a deployed position when the lancet engages with the cartridge,
wherein the one or more needles are configured to puncture the skin of the subject to draw a physiological sample when in the deployed position.

2. The dermal patch system of claim 1, wherein the cartridge is configured to store the drawn physiological sample.

3. The dermal patch system of claim 1, wherein the one or more needles includes two needles.

4. The dermal patch system of claim 1, wherein the one or more needles includes three needle.

5. The dermal patch system of claim 1, wherein the lancet includes a housing, and the one or more needles are disposed within the housing when in the undeployed position and extend beyond the housing when in the deployed position.

6. The dermal patch system of claim 5, wherein the one or more needles are configured to automatically retract into the housing after puncturing the skin.

7. The dermal patch system of claim 6, wherein the lancet further includes:
an injection spring configured to move the one or more needles to the deployed position, and
a retraction spring configured to retract the one or more needles into the housing.

8. The dermal patch system of claim 7, further comprising a mechanism for maintaining the retraction spring is a compressed state when the one or more needles are in the undeployed position and is configured to expand to retract the one or more needles.

9. The dermal patch system of claim 7, wherein the lancet further includes a plurality of deformable tabs configured to compress the retraction spring when the one or more needles are in the undeployed position.

10. The dermal patch system of claim 9, wherein the lancet further includes:
a needle platform that carries the one or more needles from the unemployed position to the deployed position,
wherein the needle platform is configured to deform or break the tabs when the lancet engages with the cartridge, thereby allowing the retraction spring to expand and cause the one or more needles to deploy.

11. The dermal patch system of claim 10, wherein the needle platform is configured to break the tabs.

12. A method for obtaining a physiological sample from a subject, comprising:
affixing a cartridge of a dermal patch system to the skin of a subject,
engaging a lancet with the cartridge, wherein the lancet includes one or more needles,
wherein engaging the lancet with the cartridge automatically moves the one or more needles from an undeployed position to a deployed position thereby drawing a physiological sample.

13. The method of claim 12, further comprising:
storing physiological sample within the cartridge.

14. The method of claim 12, further comprising:
retracting the one or more needles into the lancet, and
removing the cartridge from the skin of the subject.

15. The method of claim 14, wherein the lancet automatically retracts the one or more needles.

16. The method of claim 15, wherein the lancet includes a first spring that moves the one or more needles to the deployed position and a second spring that retracts the needles.

17. The method of claim 16, further comprising:
deforming a portion of the lancet whole moving the one or more needles to the deployed position.

18. The method of claim 17, wherein deforming a portion of the lancet allows the second spring to retract the one or more needles.

19. The dermal patch system of claim 12, wherein the one or more needles includes two needles.

20. The dermal patch system of claim 12, wherein the one or more needles includes three needles.

21. The dermal patch system of claim 1, wherein the one or more needles includes four needles.

22. The dermal patch system of claim 4, wherein the one or more needles are equally spaced from one another.

23. The dermal patch system of claim 22, wherein tips of the three needles form an equilateral triangle.

24. The dermal patch system of claim 12, wherein the one or more needles includes four needles.

25. The dermal patch system of claim 20, wherein the one or more needles are equally spaced from one another.

26. The dermal patch system of claim 25, wherein tips of the three needles form an equilateral triangle.
